(19) United States
(12) Patent Application Publication
(10) Pub. No.: US 2023/0354807 A1
(43) Pub. Date: Nov. 9, 2023

(54) SURFACE DISINFECTANT AND COATING

(71) Applicant: VERI NANO INC., Boston, MA (US)

(72) Inventors: Samson AFEWERKI, Uppsala (SE); Guillermo U. RUIZ ESPARZA HERRERA, Boston, MA (US); Joseph V. Bonventre, Wayland, MA (US); Nathan N. Lee, Boston, MA (US); Ketian ZHANG, Boston, MA (US)

(73) Assignee: VERI NANO INC., Boston, MA (US)

(21) Appl. No.: 18/246,332

(22) PCT Filed: Sep. 22, 2021
(86) PCT No.: PCT/US2021/051544

§ 371 Date: Mar. 22, 2023

Related U.S. Application Data
(60) Provisional application No. 63/149,438, filed on Feb. 15, 2021.

(30) Foreign Application Priority Data
Sep. 23, 2020 (SE) 2051106-9

(51) Int. Cl. A01N 33/12; A01N 31/08; A01N 25/10; A01N 31/02; A01N 37/36; A01N 37/02; C09D 5/14; C09D 7/63; C09D 7/20; C09D 101/28; C09D 133/04; C09D 129/04; A23B 7/16; A23L 3/349; A23L 3/3526

(52) U.S. Cl. CPC A01N 33/12; A01N 31/08; A01N 25/10; A01N 31/02; A01N 37/36; A01N 37/02; C09D 5/14; C09D 7/63; C09D 7/20; C09D 101/28; C09D 133/04; C09D 129/04; A23B 7/16; A23L 3/349; A23L 3/3526

(57) ABSTRACT

The invention relates to the film-forming antimicrobial, antibacterial, antiviral, antiparasitic, and antifungal composition comprises a) a film-forming materials, b) one or more antimicrobial, antibacterial, antiviral, antiparasitic, and antifungal compound(s), c) one or more solubilizing agents, and optionally, d) one or more plasticizer, and e), one or more surfactants or detergents. The invention also relates to a use in prevention or treatment of a disorder in a mammal, such as a human and use of treating animated and non-animated surfaces.

### FIELD OF THE INVENTION

The invention relates to the film-forming antimicrobial, antibacterial, antiviral, antiparasitic, and antifungal composition comprises a) a film-forming materials, b) one or more antimicrobial, antibacterial, antiviral, antiparasitic, and antifungal compound(s), c) one or more solubilizing agents, and optionally, d) one or more plasticizer, and e), one or more surfactants or detergents. The invention also relates to a use in prevention or treatment of a disorder in a mammal, such as a human and use of treating animated and non-animated surfaces.

### BACKGROUND

Our planet and human society are facing devastating challenges regarding global health threats from viral and bacterial infections causing tremendous healthcare costs and mortality. Similarly, fungal and parasitic infections not only affect humans and animals, but also plants and corps accounting for serious agricultural damages.

Moreover, environmental challenges are further huge problems we are encountering. In this context, the invention of innovative environmentally friendly technologies for battling these huge challenges is critically required.

Recently, we met a great taste from these challenges in the worst global pandemic, the coronavirus disease 2019 (COVID-19), caused by the novel severe acute respiratory syndrome coronavirus 2 (SARS-Cov-2), which have impacted our society tremendously leading to humanitarian and economic crises. This incident will definitely lead to a paradigm shift in our society and daily lifestyle, resulting in precaution and improved hygienic routine in various domestic and public settings. In particular, retails and hospitals, public spaces and transportations, schools and governmental agencies, food and packaging.

The general means of reducing the spreading of microorganisms and viruses are through hand washing and effective sanitizing. However, most sanitizing and disinfectant products have a short lifetime and only prevent the spreading of the microorganism or viruses, or killing these at the time of use.

There is a significant number of surface disinfectants containing components such as triclosan, however, this compound has created several human health concerns related to microbial resistance. Alcohol-based disinfectants are known to display antimicrobial activity, nevertheless, their fast evaporation limits their application. Chlorine-based disinfectants are also another class of disinfectants widely used, but they might be toxic and lead to corrosion. Quaternary ammonium-based disinfectants are also employed and have shown to display high antimicrobial activity. Nonetheless, they might be toxic and have certain limited activity against some types of microbes.

Examples of disinfectant products recommended for use against SARS-Cov-2 and other microbial organisms are, for instance, Lysol® a high concentration alcohol-based disinfectant, or Descide Ultra Disinfecting Spray that is a quaternary ammonium and alcohol-based disinfectant. Nevertheless, one of the main limitations with the commercially available disinfectant is that they only function upon application, thus effectively killing microbes immediately on the surface, for a limited amount of time, nevertheless, surfaces can easily and rapidly be re-contaminated, since the antimicrobial activity of most disinfectants does not last for longer periods of time.

Therefore, technologies that can continue protecting various surfaces for a prolonged period of time and that are sustainable would significantly reduce bacterial, viral, fungal, and parasitic spreading. Within this framework, there are very few numbers of products that can last for a long period of time, for instance, Microban®, a disinfectant formulation comprising an oxazoline homo-polymer binder and a biocidal compound. However, this technology only kills microorganisms for up to 24 h. BioShield® is another technology that provides continuous antimicrobial protection for at least 90 days comprising a coating of organosilane homopolymers providing biostatic and mechanically antimicrobial surface protection. Another technology developed by Sherwin-Williams Co. is an antimicrobial paint composition comprising a quaternary ammonium compound, however, the technology showed a capacity of killing bacteria at a rate of greater than 3 logs within 2 h of bacteria exposure.

To conclude, more than ever, our society and the planet need innovative and environmentally friendly long-lasting solutions that prevent and limit the spread of bacteria, viruses, fungal and parasitic organisms, and viruses in various public, industrial, domestic, professional, and academic settings. This might ensure better preparation for future unexpected pandemic events as well as for other diseases and health hazards.

### SUMMARY

It is an aim of the present invention to at least partly overcome the above-mentioned problems, and to provide an improved film-forming antimicrobial, antibacterial, antiviral, antiparasitic, and antifungal composition. The composition can be used as a coating.

Herein, differently from the above-presented technologies, is disclosed a dual functional environmentally friendly antibacterial, antiviral, antifungal, and antiparasitic transparent surface disinfectant, with long-lasting robust activity in the form of a coating invention. The presented invenion kills a wide range of bacteria, viruses, fungal, and parasite organisms immediately after application and continues to protect the surface and kill these organisms for several months. The present inventin prevents re-contamination of surfaces. The eco-friendly film-forming material comprises the sustainable cellulose and its derivatives or acrylate polymers. Cellulose is the most abundant renewable and sustainable material and displays countless vital characteristic advantages such as being inexpensive, biodegradable, non-toxic, and with outstanding mechanical properties. The facile fabrication approach comprises by simply mixing the film-forming material combined with biocidal components with an appropriate solvent such as ethanol, water without the use of toxic ingredients. The risk for corrosion is minimized or non-existing. The formulation can easily be applied through various application methods such as spraying or wiping onto a wide range of hard and soft, organic and inorganic surfaces and materials.

The invention relates to a film-forming antimicrobial, antibacterial, antiviral, antiparasitic, and antifungal composition, comprising,

a) a film-forming materials of one or more polysaccharide having the structural formula (I)

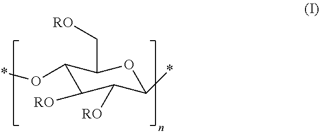

wherein, independently for each occurrence, R is selected from H or alkyl, alkenyl, alkynyl, alkoxy, allyl, cycloalkyl, heterocycle, pyridyl derivative, peptide, polypeptide, alkylamino, amine, anilino, benzyl, carboxyl, carboxyalkyl, carboxyalkenyl, cyano, glycosyl, halogen, hydroxyl, silanes, oxazolinium mesylate, oxazolinium tosylate, oxazolinium triflate, silyl oxazolinium, phenolic, polyalkoxy, quaternary ammonium groups, thiol, or thioether group or a macrocyclic structure, which are linear, branched, cyclic,

wherein said alkyl, alkenyl or alkynyl group are unsubstituted or substituted with alkyl, alkenyl, alkynyl, alkoxy, allyl, cycloalkyl, heterocycle, pyridyl derivative, peptide, polypeptide, alkylamino, amine, anilino, benzyl, carboxyl, carboxyalkyl, carboxyalkenyl, cyano, glycosyl, halogen, hydroxyl, silanes, oxazolinium mesylate, oxazolinium tosylate, oxazolinium triflate, silyl oxazolinium, phenolic, polyalkoxy, sugar, quaternary ammonium groups, thiol, or thioether group or a macrocyclic structure, which are linear, branched, cyclic, natural products and fragments thereof, polymer, polyesters, polycaprolactone, poly(lactide), poly(carboxylate), polyethylene glycol, poly(anhydride), polyamide, polysaccharides, oligomers, polyorthoester or copolymer thereof, provided at least one instance of R is not H,

wherein, n is in a range of 1 to 10,000,000, or in a range of 100 to 100,000, or 300 to 14,000, and/or

a2) a film forming materials of acrylate polymers or co-polymers,

b) one or more antimicrobial, antibacterial, antiviral, antiparasitic and antifungal compounds,

d) optionally, one or more plasticizer,

c) optionally one or more solubilizing agents, and

e) optionally one or more surfactants or detergents,

In some aspects, independently for each occurrence, R is selected from H or alkyl, alkenyl, alkynyl, alkoxy, allyl, cycloalkyl, heterocycle, pyridyl derivative, peptide, polypeptide, alkylamino, amine, anilino, benzyl, carboxyl, carboxyalkyl, carboxyalkenyl, cyano, glycosyl, halogen, hydroxyl, silanes, oxazolinium mesylate, oxazolinium tosylate, oxazolinium triflate, silyl oxazolinium, phenolic, polyalkoxy, quaternary ammonium groups, thiol, or thioether group or a macrocyclic structure, which are linear, branched, cyclic, wherein said alkyl, alkenyl or alkynyl group are unsubstituted or substituted with one or more alkyl, alkenyl, alkynyl, alkoxy, allyl, cycloalkyl, heterocycle, pyridyl derivative, peptide, polypeptide, alkylamino, amine, anilino, benzyl, carboxyl, carboxyalkyl, carboxyalkenyl, cyano, glycosyl, halogen, hydroxyl, silanes, oxazolinium mesylate, oxazolinium tosylate, oxazolinium triflate, silyl oxazolinium, phenolic, polyalkoxy, sugar, quaternary ammonium groups, thiol, or thioether group or a macrocyclic structure, which are linear, branched, cyclic, natural products and fragments thereof, polymer, polyesters, polycaprolactone, poly(lactide), poly(carboxylate), polyethylene glycol, poly(anhydride), polyamide, polysaccharides, oligomers, polyorthoester, or copolymer thereof, provided at least two instance of R is not H.

In some aspects, independently for each occurrence, R is selected from H or alkyl, alkenyl, alkynyl, alkoxy, allyl, cycloalkyl, heterocycle, pyridyl derivative, peptide, polypeptide, alkylamino, amine, anilino, benzyl, carboxyl, carboxyalkyl, carboxyalkenyl, cyano, glycosyl, halogen, hydroxyl, silanes, oxazolinium mesylate, oxazolinium tosylate, oxazolinium triflate, silyl oxazolinium, phenolic, polyalkoxy, quaternary ammonium groups, thiol, or thioether group or a macrocyclic structure, which are linear, branched, cyclic, wherein said alkyl, alkenyl or alkynyl group are unsubstituted or substituted with alkyl, alkenyl, alkynyl, alkoxy, allyl, cycloalkyl, heterocycle, pyridyl derivative, peptide, polypeptide, alkylamino, amine, anilino, benzyl, carboxyl, carboxyalkyl, carboxyalkenyl, cyano, glycosyl, halogen, hydroxyl, silanes, oxazolinium mesylate, oxazolinium tosylate, oxazolinium triflate, silyl oxazolinium, phenolic, polyalkoxy, sugar, cyclodextrins, quaternary ammonium groups, thiol, or thioether group or a macrocyclic structure, which are linear, branched, cyclic, natural products and fragments thereof, polycarbonates, polymer, polyesters, polycaprolactone, poly(lactide), poly(carboxylate), polyethylene glycol, poly(anhydride), polyamide, polysaccharides, oligomers, polyorthoester, or copolymer thereof, provided at least three instance of R is not H.

In some aspects, R is H.

In some aspects, the film forming material is selected from the group comprising or consisting of ethyl cellulose, lignocellulose, cellulose derivative such as hydroxyethyl cellulose, cellulose acetate, nanocellulose, nanofibril cellulose, nanocrystalline cellulose, and its derivative, zein, polyethylene glycol, chitin, chitosan, polyvinyl alcohol, polylactic acid, polyglycolic acid, poly lactic-co-glycolic acid (PLGA), polycaprolactone, or polysaccharide thereof. In some aspects, the film forming materials is selected from cellulose or its derivatives, physically entrapped or covalently functionalized together with b) one or more antimicrobial, antibacterial, antiviral, antiparasitic and antifungal compounds and d) one or more solubilizing agents. In some aspects, the film forming material is ethyl cellulose.

In some aspects, ethyl cellulose has a characteristic viscosity from about 4 cP to about 21000 cP, or from about 10 cP to about 500 cP, or from about 50 cP to about 450 cP, or from about 75 cP to about 400 cP, or from about 100 cP to about 400 cP, or about 300 cP (5% in toluene/ethanol 80:20).

In some aspects, the composition comprises ethyl cellulose in an amount from 0.01 wt % to 20 wt %, or from 0. 1 wt % to 15 wt %, or from 0.5 wt % to 10 wt %, or about 1.0 wt %. The percentage is the weight percentage based on the total weight of the composition.

In some aspects, the invention relates to a water insoluble film forming antimicrobial, antibacterial, antiviral, antiparasitic and antifungal composition, comprising or consisting of,

a) a film forming materials selected from the group comprising or consisting of ethyl cellulose, polyvinyl alcohol, polyvinyl acetate, polyvinyl alcohol-polyethylene glycol graft-copolymer, and/or an acrylate polymers, poly(methacrylic acid)polymer or co-polymers,

b) one or more antimicrobial, antibacterial, antiviral, antiparasitic and antifungal compounds selected from the group comprising or consisting of thymol, benzethonium chloride, benzalkonium chloride, citric acid, lactic acid, carvacrol, 2-phenylphenol, chloroxylenol, 1,2-hexanediol, 1,2-octanediol, terpineol and 1,2-decanediol,

c) one or more solubilizing agents, which is a solvent, or a mixture of solvents selected from the group comprising or consisting of water and a low molecular weight alcohol, such as ethanol and propanol, and

d) optionally, one or more plasticizer,

e) optionally, one or more surfactants or detergents.

In some aspects, the water insoluble film forming antimicrobial, antibacterial, antiviral, antiparasitic and antifungal composition, comprises or consists of

a) a film forming materials selected from the group comprising or consisting of ethyl cellulose, polyvinyl alcohol, or an acrylate polymers or co-polymers,

b) one or more antimicrobial, antibacterial, antiviral, antiparasitic and antifungal compounds selected from the group comprising or consisting of thymol, benzethonium chloride, benzalkonium chloride, citric acid, lactic acid and 1,2-hexanediol,

c) one or more solubilizing agents, which is a solvent, or a mixture of solvents selected from the group comprising or consisting of water and a low molecular weight alcohol, such as ethanol and propanol,

d) optionally, one or more plasticizer, and

e) optionally, one or more surfactants or detergents.

In some aspects, the film is a water repellent film.

In some aspects, one or more antimicrobial, antibacterial, antiviral, antiparasitic and antifungal compounds is selected from the group comprising or consisting of thymol, carvacrol, 1,2-hexanediol and 1,2-octanediol. In some aspects, one or more antimicrobial, antibacterial, antiviral, antiparasitic and antifungal compounds is thymol and/or 1,2-hexanediol.

The composition as defined anywhere herein is believed to provide advantages over the current products on the market. The film is environmentally friendly. The composition is biocompatible and biodegradable or poorly biodegradable materials. The composition is facile and fast fabrication. It has long-lasting stability and can withstand high and low temperature, long-term storage stability, friction, centrifugation-, wiping- and is stable at different UV radiations once applied as a coating on an animated or nonanimated surface. The composition has a dual action, immediate killing and subsequently formation of a long-lasting and stable film that continues protecting the surface of interest. The protection may last for 1 to 12 months, or 3 to 9 months. The composition is non-toxic, because use of strong chemicals that can be harmful to human health are avoided. The composition can be prepared at a large scale.

The coating is transparent, easy to manufacture, easy to use, stable but yet easy removable upon demand for instance by applying baking soda solution.

A coating with an acrylate polymers or co-polymers is water-resistant and transparent.

In some aspects, the film forming material is a dispersion.

In some aspects, the acrylate polymers or co-polymers is selected from the group comprising or consisting of methacrylic acid-ethyl acrylate copolymer and methacrylic acid-acrylic ester copolymer. In some aspects, the acrylate polymers or co-polymers is methacrylic acid-ethyl acrylate copolymer. In some aspects, the acrylate polymers or co-polymers is methacrylic acid-ethyl acrylate copolymer. In some aspects, the acrylate polymers or co-polymers is Kollicoat MAE 30 DP™.

A coating with an acrylate polymers or co-polymers is water-resistant and transparent. Cellulosic compositions, e.g. wherein the film forming materials contains ethyl cellulose or hydroxyethyl cellulose or methyl cellulose, may have a disadvantage of not being transparent when the droplet size is too large when spraying the composition on a surface.

In some aspects, the acrylate polymers or co-polymers or methacrylic acid-ethyl acrylate copolymer is in a weight ratio of 0.1:99 to 99:1, or 1:9 to 9:1, or 8:2 to 2:8, or 6:4 to 4:6, preferable 1:1. In some aspects, the acrylate polymers or co-polymers or methacrylic acid-ethyl acrylate copolymer is in a volume ratio of 0.1:99 to 99:1, or 1:9 to 9:1, or 8:2 to 2:8, or 6:4 to 4:6, preferable 1:1. In some aspects, the film forming materials is methacrylic acid-ethyl acrylate copolymer at a weight or volume ratio for methacrylic acid to ethyl acrylate of 5:1 to 1:5, or 2:1 to 1:2.

In some aspects, the pH of the liquid is below 6, or below 5. In some aspects, the film forming material is an acrylate polymers or co-polymers or methacrylic acid-ethyl acrylate copolymer and the pH of the liquid is below 6, or below 5 or below 4.

In some aspects, the viscosity of the liquid is from 1 to 2, or from 1.2 to 1.5, or from 1.3 to 1.5, or about 1.407 mPa/s at 25° C., as measured on a TA Instrument DHR-2 Rheometer. In one aspect, liquid comprises methacrylic acid-ethyl acrylate copolymer.

In some aspects, the water insoluble film forming antimicrobial, antibacterial, antiviral, antiparasitic and antifungal composition comprises or consisting of

a) a film forming materials selected from methacrylic acid-ethyl acrylate copolymer at a ratio of 1:2 to 2:1,

b) one or more antimicrobial, antibacterial, antiviral, antiparasitic and antifungal compounds selected from the group comprising or consisting of thymol, 1,2-hexanediol and 1,2-octanediol,

c) one or more solubilizing agents, which is a solvent or a mixture of solvents selected from the group comprising or consisting of water and ethanol,

d) optionally, one or more plasticizer, and

e) optionally, one or more surfactants or detergents.

In some aspects, the water insoluble film forming antimicrobial, antibacterial, antiviral, antiparasitic and antifungal composition comprises or consisting of

a) a film forming materials selected from methacrylic acid-ethyl acrylate copolymer at a ratio of 1:1,

b) one or more antimicrobial, antibacterial, antiviral, antiparasitic and antifungal compounds selected from the group comprising or consisting of thymol and 1,2-hexanediol,

c) one or more solubilizing agents selected from water and ethanol,

d) optionally, one or more plasticizer, and

e) optionally, one or more surfactants or detergents selected from polysorbate 80 and/or sodium lauryl sulfate.

In some aspects, the water insoluble film forming antimicrobial, antibacterial, antiviral, antiparasitic and antifungal composition comprises or consisting of

a) a film forming materials selected from Kollicoat MAE 30 DP™,

b) one or more antimicrobial, antibacterial, antiviral, antiparasitic and antifungal compounds selected from the group comprising or consisting of thymol and 1,2-hexanediol,

c) one or more solubilizing agents selected from water and ethanol.

In some aspects, the one or more antimicrobial, antibacterial, antiviral, antiparasitic and 35 antifungal compound is selected from the group comprising or consisting of benzethonium chloride, benzalkonium chloride, thymol, lactic acid, chitosan, triethylene glycol, diols, 1,2-hexanediol, octanoic acid, sodium dichloroisocyanurate, citric acid, caffeic acid, caffeic acid-Fe (III) chelate, quercetin-3-rutinoside (rutin), capsaicin, antimicrobial peptides, polylysine, polyethyleneimine, polyguanidines, phenolic or polyphenols, such as lignin and tannin, lycorine, chitosan, carbon nanotubes, fullerene, graphene, graphene oxide, peroxides, β-caryophyllene, star anise oil, berberine chloride, palmatine, novobiocin, morin, polyamines, phenols, triclosan, dimethylamine, epichlorohydrin, superfloc C-572, quercetin, glycyrrhizic acid, sulfamic acid, glycolic acid, lauric acid, capric acid, or any mixture thereof, quaternary ammonium based compounds including, but are not limited to n-alkyl dimethylbenzyl ammonium chloride, di-n-octyl dimethylammonium chloride, dodecyl dimethyl ammonium chloride, n-alkyl dimethylbenzyl ammonium saccharinate, and 3-(trimethoxysilyl) propyldimethyloctadecyl ammonium chloride, poly diallyldimethylammonium chloride, poly DADMAC, polyhexamethylene biguanide (PHMB), chlorhexidine, Bardac® 205M, 208M and BTC885, quaternary ammonium based silanes such as 3(trihydroxysilyl) propyldimethyloctadecyl ammonium chloride, or any mixture thereof, metal based such as metal salts, metal nano- and microparticles, and metallics compounds, including, but are not limited to zinc, zinc pyrithione, silver, silver nanoparticles, cerium, cerium oxide nanoparticles, selenium nanoparticles, copper nanoparticles, copper, gold, gold nanoparticles, titanium, titanium nanoparticles, iron, iron nanoparticles, platinum-, tin-, ruthenium-, cobalt-based antibacterial, antiviral, antifungal, and antiparasitic compounds, or any mixture thereof.

In some aspects, the antimicrobial, antibacterial, antiviral, antiparasitic and antifungal compound comprises or consists of one or more compounds selected from the group benzethonium chloride, benzalkonium chloride and thymol.

In some aspects, the antimicrobial, antibacterial, antiviral, antiparasitic and antifungal compound comprises or consists of a combination of benzethonium chloride, benzalkonium chloride and thymol.

In some aspects, the one or more antimicrobial, antibacterial, antiviral, antiparasitic and antifungal compound is thymol.

In some aspects, the one or more antimicrobial, antibacterial, antiviral, antiparasitic and antifungal compound is 1,2-hexanediol.

In some aspects, the one or more antimicrobial, antibacterial, antiviral, antiparasitic and antifungal compound is selected from the group comprising or consisting of thymol and 1,2-hexanediol. In some aspects, film forming materials is an acrylate polymers or co-polymers selected from the group comprising methacrylic acid-ethyl acrylate copolymer and methacrylic acid-acrylic ester copolymer, and the one or more antimicrobial, antibacterial, antiviral, antiparasitic and antifungal compound is selected from the group comprising or consisting of thymol and 1,2-hexanediol.

In some aspects, the one or more antimicrobial, antibacterial, antiviral, antiparasitic and antifungal compound is 1,2-octanediol. In some aspects, the one or more antimicrobial, antibacterial, antiviral, antiparasitic and antifungal compound is selected from the group comprising or consisting of thymol and 1,2-octanediol. In some aspects, film forming materials is an acrylate polymers or co-polymers selected from the group comprising methacrylic acid-ethyl acrylate copolymer and methacrylic acid-acrylic ester copolymer, and the one or more antimicrobial, antibacterial, antiviral, antiparasitic and antifungal compound is selected from the group comprising or consisting of thymol and 1,2-octanediol.

Thymol and 1,2-hexanediol are safe for human and none human use. These compound are already being used in human products. Thymol is highly effective in killing microbes. Thymol also has a pleasant odor that can mask the odors of Kollicoat MAE 30 DP and 1,2-hexanediol. Though the antimicrobial efficacy of 1,2-hexanediol is low, it can act in synergy with other antimicrobial agents (see US20070265352A1). Thymol is highly effective in reducing the minimum inhibitory concentration (MIC) of several antibiotics, and also act synergistically with antibiotics and other agents promoting the overall antimicrobial efficacy. The methacrylic acid-ethyl acrylate copolymer is also antibacterial (killing 78% against E. coli in 10 min) besides acting as a film-forming material.

In some aspects, the one or more antimicrobial, antibacterial, antiviral, antiparasitic and antifungal compound combination comprises or consists of an amount from 0.01 w/v % to 20 w/v %, or 0.01 w/v % to 10 w/v %, or 0.05 w/v % to 5 w/v %, or 0.1 w/v % to 1 w/v %, or about 0.2 w/v % of benzethonium chloride, from 0.01 w/v % to 1 w/v%, or about 0.1 w/v % of benzalkonium chloride and from 0.01 w/v % to 1 w/v %, or about 0.1 w/v % of thymol. The percentage is the weight/volume percentage based on the total volume of the solvent. In some aspects, the concentrations of the antimicrobial, antibacterial, antiviral, antiparasitic and antifungal compound is 0.01-0.8 v/v % of 1,2-hexanediol and 0.01-0.8 w/v % of thymol, or 0.1-0.5 v/v % of 1,2-hexanediol and 0.1-0.5w/v % of thymol, or 0.1-0.3 v/v % of 1,2-hexanediol and 0.1-0.3w/v % of thymol, or 0.25-3.2v/v% 1,2-hexanediol and 0.15-0.25 w/v % thymol (concentrations are relative to the solvent (water/ethanol mixture) w/v for solids and v/v for liquids).

In some aspects, the concentrations of the antimicrobial, antibacterial, antiviral, antiparasitic and antifungal compound is 0.25 v/v % 1,2-hexanediol and 0.20 w/v % thymol. In some aspects, the concentrations of the antimicrobial, antibacterial, antiviral, antiparasitic and antifungal compound is 0.30 v/v % 1,2-hexanediol and 0.24 w/v % thymol.

In some aspects, the plasticizer is selected from the group comprising or consisting of glyceryl trioctanoate, dibutyl sebacate, triethyl citrate, triacetin, Myvacet® (acetylated monoglycerides) and diethyl phthalate.

In some aspects, the plasticizer is glyceryl trioctanoate.

In some aspects, the plasticizer comprises glyceryl trioctanoate present in an amount from 0.01 wt % to 20 wt %, or 0.01 wt % to 10 wt %, or 0.05 wt % to 5 wt %, or 0.1 wt % to 1 wt %, or 0.01 wt % to 1 wt %, or about 0.1 wt %.

In some aspects, the plasticizer is selected from the group comprising or consisting of 1,2-hexanediol, glycerol, caprylic/capric triglycerides, glycerol monolaurate, or a combination thereof, especially when the film forming materials is an acrylate polymers or co-polymers.

In some aspects, the solubilizing agent comprises a solvent or a mixture of solvents.

In some aspects, the solvent or a mixture of solvents is selected from the group comprising or consisting of water, a low molecular weight alcohol, alkylene glycol ether based solvents, including but are not limited to, ethylene glycol monopropyl ether, ethylene glycol monobutyl ether, ethylene glycol monohexyl ether, ethylene cyclo monohexyl ether, diethylene glycol monomethyl ether, diethylene glycol monoethyl ether, diethylene glycol monobutyl ether, diethylene glycol monohexyl ether, triethylene glycol monomethyl ether, triethylene glycol monoethyl ether, triethylene glycol monobutyl ether, propylene glycol methyl ether, propylene glycol methyl ether acetate, propylene glycol n-butyl ether, dipropylene glycol n-butyl ether, dipropylene glycol methyl ether, dipropylene glycol methyl ether acetate, propylene glycol n-propyl ether, dipropylene glycol n-propyl ether, and tripropylene glycol methyl ether, butanol, pentanol, acetone, isopropanol, ethyl acetate, glycerol, essential oils, pine oil, lemon oil, limonene, pinene, cymene, myrcene, fenchone, borneol, nopol, cineol, ionone, inorganic buffer, buffer solution, a terpene and terpene derivative, or any mixture thereof.

In some aspects, the solvent or a mixture of solvents is selected from the group comprising water, a low molecular weight alcohol such as ethanol, or any mixture thereof, especially when the film forming materials is an acrylate polymers or co-polymers.

In some aspects, the solvent is selected from pure ethanol or denatured ethanol. In some aspects, the solvent is a mixture of ethanol and isopropanol.

In some aspects, the solvent is a mixture of water and ethanol. The amount of ethanol in the final composition may be 5 to 50 wt %, or 5 to 25 wt %, or 5 to 20 wt %, 5 to 15 wt %, or 10 to 20 wt %, or 10 to 15 wt %.

These solvents are easily available at low costs, environmentally friendly and evaporate quick enough (i.e. within minuts. The solvent is suitable for large scale production of the composition. The fast evaporation of the solvents promotes the fast film formation.

In some aspects, the surfactant or detergents or a mixture of surfactants or detergents, which may be anionic, is selected from the group comprising or consisting of glyceryl trioctanoate, dibutyl sebacate, triethyl citrate, triacetin, myvacet® (acetylated monoglycerides) and diethyl phthalate or 1,2-hexanediol, glycerol, caprylic/capric triglycerides, glycerol monolaurate, anionic detergent and surfactant such as sodium lauryl sulfate, alkylbenzene sulfonates, deoxycholic acid, sulfates include ammonium lauryl sulfate, (sodium dodecyl sulfate, sls, or sds), and the related alkyl-ether sulfates sodium laureth sulfate (sodium lauryl ether sulfate or sles), and sodium myreth sulfate, docusate (dioctyl sodium sulfosuccinate), perfluorooctanesulfonate (pfos), perfluorobutanesulfonate, alkyl-aryl ether phosphates, alkyl ether phosphates, carboxylates such as sodium stearate, sodium lauroyl sarcosinate, carboxylate-based fluorosurfactants such as perfluorononanoate, perfluorooctanoate (pfoa or pfo), or selected from the group comprising nonionic and zwitter ionic surfactants and detergens such as polysorbate 80, polyoxyethylene, glycoside, ethoxylates, fatty alcohol ethoxylates, narrow-range ethoxylate, octaethylene glycol monododecyl ether, pentaethylene glycol monododecyl ether, alkylphenol ethoxylates (apes or apeos), nonoxynols, triton x-100, fatty acid ethoxylates, special ethoxylated fatty esters and oils, ethoxylated amines and/or fatty acid amides, polyethoxylated tallow amine, cocamide monoethanolamine, cocamide diethanolamine, terminally blocked ethoxylates, poloxamers, fatty acid esters of polyhydroxy compounds, fatty acid esters of glycerol, glycerol monostearate, glycerol monolaurate, fatty acid esters of sorbitol, sorbitan monolaurate, sorbitan monostearate, sorbitan tristearate, tweens: tween 20, tween 40, tween 60, tween 80, fatty acid esters of sucrose, alkyl polyglucosides, alkyl polyglycoside such as decyl glucoside, lauryl glucoside, octyl glucoside, or cationinc detergents and surfactants, such as octenidine dihydrochloride, cetrimonium bromide (ctab), cetylpyridinium chloride (cpc), benzalkonium chloride (bac), benzethonium chloride (bzt), dimethyldioctadecylammonium chloride, and dioctadecyldimethylammonium bromide (dodab).

In some aspects, the surfactant or detergents or a mixture of surfactants or detergents is polysorbate 80 and/or sodium lauryl sulfate. In some aspects, film forming materials is an acrylate polymers or co-polymers selected from the group comprising or consisting of methacrylic acid-ethyl acrylate copolymer and methacrylic acid-acrylic ester copolymer, and the surfactant or detergents or a mixture of surfactants or detergents is polysorbate 80 and/or sodium lauryl sulfate.

In some aspects, the water insoluble film forming antimicrobial, antibacterial, antiviral, antiparasitic and antifungal composition further comprises one or more odour masking agent, optionally selected from the group comprising or consisting of menthol, citronellal, citrus, lemon, fragrances, Fragrance Blood Orange, Fragrance Pink Grapefruit Passion Fruit, Fragrance Mangosteen, Fragrance Coral Reef, Fragrance Citrus Punch, lemon fragance, and orange fragance and lavender fragrance.

In some aspects, the water insoluble film forming antimicrobial, antibacterial, antiviral, antiparasitic and antifungal composition further comprises one or more odour masking agent, optionally selected from the group comprising or consisting of menthol, citronellal, citrus, lemon, fragrances, such as Fragrance Lemon Verbena (Making Cosmetics Lot #071118MCLV) (preferable concentrations 0.05%, 0.1%, 0.2%, 0.5%, and 1%, or 0.1% or 0.2%), Fragrance Blood Orange (Making Cosmetics Lot #171101015), Fragrance Pink Grapefruit Passion Fruit

(Making Cosmetics Lot #180728016), Fragrance Mangosteen (Making Cosmetics Lot #180620050), Fragrance Coral Reef (Making Cosmetics Lot #170815034), Fragrance Citrus Punch (Making Cosmetics Lot #180206062), lemon fragance, and orange fragance and lavender fragrance.

In some aspects, the water insoluble film forming antimicrobial, antibacterial, antiviral, antiparasitic and antifungal composition further comprises one or more anti itching agent, optionally selected from the group comprising or consisting of pramoxine hydrochloride, diphenhydramine, cetirizine, loratadine, desloratadine and fexofenadine.

In some aspects, water insoluble film forming antimicrobial, antibacterial, antiviral, antiparasitic and antifungal composition further comprises or consists of one or more anti-inflammatory agent, optionally selected from the group comprising ibuprofen, naproxen, diclofenac, celecoxib, mefenamic acid, etoricoxib, indomethacin, aspirin, diflunisal and neosporin.

In some aspects, the composition is in the form of a transparent liquid solution that upon spraying onto surfaces forms a stable and transparent coating.

Some aspects of the invention relate to a use of the film forming antimicrobial, antibacterial, antiviral, antiparasitic and antifungal composition as defined herein, as a coating.

The invention relates to a coating made of or comprising or consisting of the film forming antimicrobial, antibacterial, antiviral, antiparasitic and antifungal composition as defined herein. In some aspects, the coating is transparent for light. In another aspect, the coating has been sprayed on a surface of an article.

In some aspects, the solution is stable and can withstand; a temperature storage at −20 and 60° C.,

a long-term storage stability at room temperature, and/or

a centrifugation at 20, 000 rcf for 1 h.

In some aspects, the coated film is stable and can withstand;

a 50 times finger swipes and 20 seconds rising with water,

a friction stability by 1,200 times cloth wipes and 1,200 times finger wipes,

a temperature stability at −20 and 60° C. for more than 24 h,

an UV-light stability for more than 1 h,

a spitting and wiping, and/or

a treatment with Lysol® disinfectant spray and wiping.

In some aspects, the composition in solution form provides a killing level of log about 0.6 or greater within seconds or log about 2.2 or greater within 10 min against a target of E. Coli and other microbes.

In some aspects, the composition after film formation provides a killing level of log about 1.6 or greater after 5 min or log about 2.1 or greater after 28 and 90 days or longer against a target of E. coli.

The invention also relates to an article treated with the film forming antimicrobial, antibacterial, antiviral, antiparasitic and antifungal composition as defined herein.

The invention also relates to a use of the film forming antimicrobial, antibacterial, antiviral, antiparasitic and antifungal composition as defined herein, by coating nonanimated surfaces selected from the group comprising nuts, meat, beef, fish, bone, trees, fruits, plants, flowers, crops, harvest plants, plastics, metal, steel, wood, ceramics, tissue, glass, granite, stone, marble, cloths, and fabrics.

In some aspects, the article has a surface made of materials selected from the group comprising or consisting of any inorganic and organic surface made from various materials, such as plastics, metal, steel, wood, ceramics, tissue, glass, granite, stone, marble, clothes, fabrics, hard and soft surfaces. These surfaces can be found in private and public spaces, forest, retails, hospitals, hotels, boats, airports, packages, boxes, bin, toilets, bathrooms, domestic, professional settings, or any mixture thereof.

The invention further relates to a use of the film forming antimicrobial, antibacterial, antiviral, antiparasitic and antifungal composition as defined herein, for coating any organic surface, such as fruits, vegetables, nuts, meat, beef, fish, bone, or any edible product, or any mixture thereof. The invention further relates to a use of the film forming antimicrobial, antibacterial, antiviral, antiparasitic and antifungal composition as defined herein, for coating foods, fruits and vegetables. The invention further relates to a use of the film forming antimicrobial, antibacterial, antiviral, antiparasitic and antifungal composition as defined herein, for coating fruits and vegetables. An example of a fruit may be an apple or an orange. The composition acts as a disinfectant, and increases the shelf-life of edible products and prevent microbial deposition, growth, spread and survival. The composition protects against external damage of chemicals, physical, and biological type. Further chemical damage includes exposure to gases, moisture, oxygen and light; physical damage refers to any damages caused by any shock or vibration; and biological damage may be caused by the action of pathogens, insects, animals, or the senescence of the food itself.

In some aspects, the use of the film forming antimicrobial, antibacterial, antiviral, antiparasitic and antifungal composition as defined herein is for coating any type of trees, fruits, plants, flowers, crops, harvest plants as a pesticide or to prevent microbial deposition, growth, spread and survival.

Some aspects related to the film forming antimicrobial, antibacterial, antiviral, antiparasitic and antifungal composition as defined herein, for use in therapy, such as for use in prevention or treatment of a disorder in a mammal, such as a human.

Some aspects related to the film forming antimicrobial, antibacterial, antiviral, antiparasitic and antifungal composition as defined herein, for use in therapy, such as for use in prevention or treatment of a skin disorder in a mammal, such as a human.

Some aspects related to the film forming antimicrobial, antibacterial, antiviral, antiparasitic and antifungal composition as defined herein, for use in prevention or treatment of a disorder in a mammal, such as a human, by coating animated surfaces, selected from human and animal tissues, such as skin, mucosa, bones, nails, hair, intraperitoneal tissues, intracranial tissues, intrapelvic tissues, intramediastinal tissues, intrapericardial tissues, during any type of surgical procedure, surgical wounds, skin wounds, mucosal wounds, skin diseases (eczema, acne vulgaris, acne rosacea), topic infectious diseases, burn injuries. Some aspects related to the film forming antimicrobial, antibacterial, antiviral, antiparasitic and antifungal composition as defined herein, for use in prevention or treatment of a disorder in a mammal, such as a human, by coating skin, hair and/or nails.

Some aspects related to the film forming antimicrobial, antibacterial, antiviral, antiparasitic and antifungal composition as defined herein, for use in a cosmetic product.

A coating formed by the composition as defined herein is believed to promote wound healing and to prevent or treat antimicrobial deposition, growth, spread and survival on human or animal tissue.

In some aspects, the use of the film forming antimicrobial, antibacterial, antiviral, antiparasitic and antifungal composition as defined herein is for coating home settings, private and public spaces, retails, hospitals, hotels, amusement parks, recreational parks, educational facilities such as schools and universities, factories, boats, ships interiors and exteriors, airports, planes interiors and exteriors, aircraft interiors and exteriors, metro and train interiors and exteriors, buses interiors and exteriors, cars interiors and exteriors, packages, boxes, bin, toilets, bathrooms, domestic, professional settings or any mixture thereof.

In some aspects, the use of the film forming antimicrobial, antibacterial, antiviral, antiparasitic and antifungal composition as defined herein is for killing one or more microbials selected from the group comprising or consisting of S. aureus, E. coli, Pseudomonas aeruginosa, Methicillin-resistant Staphylococcus aureus (MRSA), S. epidermidis, enterobacter cloacae, enterococcus faecalis, corynebacterium, clostridium, listeria, bacillus, S. saprophyticus, S. pyogenes, S. agalactiae, Saccharomyces cerevisiae, Aspergillus brasiliensis, S. pneumoniae, S. viridans, salmonella, shigella, moraxella, helicobacter, stenotrophomonas, bdellovibrio, legionella, Neisseria, gonorrhoeae, Neisseria meningitidis, moraxella catarrhalis, haemophilus influenzae, proteus mirabilis, enterobacter cloacae, serratia marcescens, helicobacter pylori, salmonella enteritidis, salmonella typhi, acinetobacter baumannii, and mycobacterium tuberculosis, and viruses, non-enveloped and enveloped viruses, selected from the group comprising coronavirus such as SARS-Cov-1 and SARS-Cov-2, SARS, Middle East respiratory syndrome (MERS), influenza A, B and C virus, hantavirus, Anders hantavirus, human parainfluenza viruses, respiratory syncytial virus, marburg virus, ebola virus, rabies, human immunodeficiency virus (HIV), smallpox, dengue, rotavirus A, B and C, human papillomaviruses (HPVs), Epstein-Barr virus (EBV), hepatitis A, B and C virus, human herpes virus (HHV), human T-lymphotropic virus-1 (HTLV-1), merkel cell polyomavirus (MCV), Equine Arteritis virus, simian virus 40 (SV40), adeno-associated virus, aichi virus, measles virus and yellow fever virus, mold, mildew, as well as fungi or parasites selected from the group comprising protozoa, nematodes, necrotrophs, biothrophs, and worms such as chrysomyxa ledi, phragmidium, taphrina confusa, epichloe typhina, armillaria, striga, albugo candida, plasmodiophora brassicae, powdery mildews, downy mildews, pythium species, sclerotinia rots (S. sclerotiorum, S. minor, sclerotium rolfsii and S. cepivorum), fusarium wilts and rots (various fusarium species including F. solani and F. oxysporum), botrytis rots (botrytis cinerea), anthracnose (colletotrichum spp. and microdochium panattonianum), rhizoctonia rots (Rhizoctonia solani), damping-off (pythium, rhizoctonia, phytophthora, fusarium or aphanomyces), cavity spot (pythium sulcatum), tuber diseases, rusts, such as puccinia sorghi, uromyces appendiculatus, puccinia allii, black root rot, alternaria solani, aphanomyces euteiches pv. phaseoli, aschochyta collar rot, didymella bryoniae, alternaria cucumerina and A. alternata, leptosphaeria maculans, mycosphaerella brassicicola, septoria apiicola, cercospora beticola, septoria petroelini, septoria lactucae, stemphylium vesicarium, alternaria dauci, candida, ringworm, blastomyces, coccidioides, histoplasma, cryptococcus gattii, neoformans, mucormycetes, cryptococcus paracoccidioides, aspergillus, taloromyces, pneumocystis jirovecii, candida auris, Candida albicans, Sporothrix, trichophyton rubrum, dermatophytes that cause tinea cruris, tinea corporis, tinea pedis, tinea versicolor, tinea unguium, tinea faciei, tinea manuum, and tinea capitis in mammals such as trichophyton rubrum, trichophyton tonsurans, trichophyton mentagrophytes, trichophyton verrucosum, trichophyton schoenlenii, trichophyton equinum, trichophyton kanei, trichophyton raubitschekii, trichophyton violaceum, microsporum canis, microsporum audouinii, microsporum gypseum, microsporum equinum, microsporum nanum, microsporum versicolor, and epidermophyton floccosum, and archaea, algae and germs, or any combinations thereof.

In some aspects, the film forming antimicrobial, antibacterial, antiviral, antiparasitic and antifungal composition as defined herein is used for killing S. aureus, E. coli, Saccharomyces cerevisiae and/or Aspergillus brasiliensis. In some aspects, the film forming antimicrobial, antibacterial, antiviral, antiparasitic and antifungal composition as defined herein is used for killing Equine Arteritis Virus, Candida auris, SARS-Cov-1 and 2. These viruses all belongs to the same class of viruses called envelop virus, that are characterized by having an outerwrapping envelope.

The invention relates to a method of treating or preventing bacteria, virus, fungal and parasitic deposition, formation, spreading and survival on surfaces, comprising the film forming antimicrobial, antibacterial, antiviral, antiparasitic and antifungal composition as defined herein, to kill and prevent bacteria, virus, fungal and parasitic deposition, formation, spreading and survival in/on surfaces.

The invention also relates to a method of treating or preventing insect's deposition, formation, spreading and survival on surfaces, comprising the film forming antimicrobial, antibacterial, antiviral, antiparasitic and antifungal composition as defined herein, to kill and prevent insect deposition, formation, spreading and survival in/on surfaces.

In some aspects, treating or preventing occurs by an application method selected from the group consisting of spraying, pressurized aerosol, fogging, rolling, brushing, mopping, wiping, dipping, injecting or any mixture thereof.

In some aspects, the film forming antimicrobial, antibacterial, antiviral, antiparasitic and antifungal composition, optionally further comprises or consists of antimicrobial, antibacterial, antiviral, antiparasitic and antifungal drugs selected from the group comprising or consisting of penicillin's such as penicillin, penicillin G, hetacillin potassium, cloxacillin benzathine, ampicillin and amoxicillin trihydrate, aminocoumarins such as novobiocin, cephalosporins such as cephalexin, ceftiofur sodium, ceftiofur hydrochloride, ceftiofur crystalline free acid, macrolides such as tildipirosin, tylosin, tulathromycin, erythromycin, clarithromycin, and azithromycin, quinolones and fluoroquinolones such as enrofloxacin, ciprofloxacin, levofloxacin, and ofloxacin, sulfonamides such as sulfadimethoxine, co-trimoxazole and trimethoprim, retinoids such as retinol, retinal, tretinoin (retinoic acid), isotretinoin, alitretinoin, etretinate, adapalene, bexarotene, and tazarotene, trifarotene, tetracyclines such as tetracycline, oxytetracycline and doxycycline, aminoglycosides such as dihydrostreptomycin sulfate, neomycin, gentamicin and tobramycin, lincosamides such as pirlimycin hydrochloride, lincomycin, clindamycin, and pirlimycin, and amphenicols such as florfenicol, antiviral drugs such as peramivir, zanamivir, oseltamivir phosphate, baloxavir marboxil, acyclovir, valacyclovir, valganciclovir, brivudin, cidofovir, famciclovir, fomivirsen, foscarnet, ganciclovir, penciclovir, amantadine, rimantadine, ribavirin, telbivudine, adefovir, entecavir, emtricita bine, lamivudine, tenofovir, boceprevir, telaprevir, interferons, antimycotic drug is selected from polyenes such as amphotericin b, candicidin, filipin, hamycin, natamycin, nystatin, and rimocidin; azoles such as imidazole, triazole, thiazole, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, and abafungin; allylamines such as amorolfin, butenafine, naftifine, and terbinafine; echinocandins such as anidulafungin, caspofungin and micafungin; aurones, benzoic acid, ciclopirox olamine, flucytosine or 5-fluorocytosine, griseofulvin, haloprogin, tolnaftate, undecylenic acid, triacetin, crystal violet, castellani's paint, orotomide (f901318), miltefosine, potassium iodide, coal tar, copper(ii) sulfate, selenium disulfide, sodium thiosulfate, piroctone olamine, iodoquinol, clioquinol, acrisorcin, zinc pyrithione, and sulfur, antiprotozoals such as melarsoprol, eflornithine, metronidazole, tinidazole, miltefosine, antihelminthics such as mebendazole, pyrantel pamoate, thiabendazole, diethylcarbamazine, ivermectin, aticestodes such as niclosamide, praziquantel, albendazole, antitrematodes such as praziquantel, antiamoebics such as rifampin and amphotericin B, and broad-spectrum drugs such as nitazoxanide, or a combination thereof.

In some aspects, the film forming antimicrobial, antibacterial, antiviral, antiparasitic and antifungal composition, optionally further comprises or consists of vitamins selected from the group comprising or consisting of retinol (vitamin A), thiamine (vitamin B1), riboflavin (vitamin B2), niacin (vitamin B3), pantothenic acid (vitamin B5), pyridoxine (vitamin B6), biotin (vitamin B7), folic acid (vitamin B9), ascorbic acid (vitamin C), ergocalciferol (vitamin D1), and tocopherols (vitamin E) or a combination thereof for medical, nutritional or cosmetic applications.

In some aspects, the film forming antimicrobial, antibacterial, antiviral, antiparasitic and antifungal composition, optionally further comprises or consists of minerals selected from the group comprising or consisting of calcium, phosphorus, potassium, sulfur, sodium, chloride, magnesium, iron, zinc, copper, manganese, iodine, cobalt, selenium, molybdenum, chromium, and fluoride or a combination thereof for medical, nutritional or cosmetic applications.

In some aspects, the film forming antimicrobial, antibacterial, antiviral, antiparasitic and antifungal composition, optionally further comprises or consists of an enzyme, an antibody, a transmembrane protein, a cell signaling proteins, and a structural protein wherein the structural protein is selected from collagen, hyaluronan, elastin, and tropoelastin or a combination thereof for medical, nutritional or cosmetic applications.

In some aspects, the film forming antimicrobial, antibacterial, antiviral, antiparasitic and antifungal composition, optionally further comprises or consists of alpha-linolenic acid (an omega-3 fatty acid), linoleic acid (an omega-6 fatty acid), docosahexaenoic acid (an omega-3 fatty acid) and gamma-linolenic acid (an omega-6 fatty acid) or a combination thereof for medical, nutritional or cosmetic applications.

In some aspects, the film forming antimicrobial, antibacterial, antiviral, antiparasitic and antifungal composition, optionally further comprises or consists of insecticides selected from the group comprising organochlorides, organophosphates, carbamates, pyrethroids, neonicotinoids, butenolides, ryanoids or a combination thereof.

In some aspects, the film forming antimicrobial, antibacterial, antiviral, antiparasitic and antifungal composition, the composition as defined herein can optionally be composed of a combination of zero, one or more of the antivirals, antibacterial, antiparasitic, antifungal drug, mineral, vitamin, fatty acid, protein and insecticidal compounds.

In some aspects, the film forming antimicrobial, antibacterial, antiviral, antiparasitic and antifungal composition, the composition as defined herein can optionally be composed of a combination of zero, one or more of the antivirals, antibacterial, antiparasitic, antifungal drug, mineral, vitamin, fatty acid, protein, odour masking agent, anti-itching agent, anti-inflammatory agent, and insecticidal compounds.

The disinfectant and long-lasting coating is able to optionally release in a sustained manner overtime antibacterial, antiviral, antifungal, antiparasitic, insecticidal, vitamin, mineral, fatty acid, odour masking, anti-itching, anti-inflammatory or protein compounds or drugs or a combination thereof.

In some aspects, the composition as defined herein can be applied on surfaces selected from the group comprising or consisting of inert surfaces of objects, clothes, equipment, vehicles and construction structures, as well as to organic materials and live organisms such as plants, humans and animals.

Some aspects of the invention relate to a use of the film forming antimicrobial, antibacterial, antiviral, antiparasitic and antifungal composition for disinfecting surfaces for treating or preventing COVID-19, or for coating fruits e.g. appels to increase their shelf-life, or for coating fruits to increase their nutritional value with encapsulated nutrients, or for coating pesticides to protect crops, or for coating crops to give the plants nutrients that will be encapsulated in the coating. This could improve plants growth and thus increase revenues for the agricultural industry.

Some aspects of the invention relate to a use of the film forming antimicrobial, antibacterial, antiviral, antiparasitic and antifungal composition for treating skin diseases by releasing agents like drugs and vitamins over time.

Some aspects of the invention relate to a use of the film forming antimicrobial, antibacterial, antiviral, antiparasitic and antifungal composition for treating or preventing skin by coating the skin and releasing nutrients, like vitamins, minerals, anti-itching agent, anti-inflammatory agent and proteins for the skin.

Furthermore, the presented inventions display good stability in liquid form and as coated film and can withstand high and low temperature, long-term storage (1-12 months, or 2-9 months, or 1 to 6 months) and various manipulations, such as centrifugation, finger and cloth wiping, rinsing with water and against Ultraviolet (UV)-light etc.

The invention also relates to a method of removing a coating made from the film forming antimicrobial, antibacterial, antiviral, antiparasitic and antifungal composition as defined herein, comprising or consisting of the steps;
applying an aquous solution comprising or consisting of one or more salt on the coating,
wiping off the coating from the coated surface.

In some aspects, the concentration of sodium bicarbonate may be from 0.05 to 5, or 1 to 2, or about 1.5 g/100 ml water.

In some aspects, the salt is selected from the group comprising or consisting of sodium bicarbonate, sodium carbonate, potassium bicarbonate, potassium carbonate, trisodium phosphate, disodium phosphate, monosodium phosphate, tripotassium phosphate, dipotassium phosphate, monopotassium phosphate, borax and phosphate-buffered saline. In some aspects, the salt is PBS (phosphate-buffered saline) or sodium bicarbonate.

### BRIEF DESCRIPTION OF THE DRAWINGS

FIG. 1 is a schematic demonstrating the step by step procedure of the culture method used for the evaluation and quantification of the antibacterial activity from the devised formulation. The steps comprising (1) Dip a swab in bacteria suspension; (2) Introduce bacteria to the surface; (3) Collect the bacteria from the surface; (4) Suspend the bacteria in phosphate-buffered saline (PBS) and (5) culture the bacteria, then count the colonies.

FIG. 2 Demonstrates some selected examples photos of the colony counting on agar plates from diluted bacteria suspensions, where the Escherichia coli (E. coli) was added at day 28 onto the coating and uncoated surface (control) and subsequently, the antibacterial activity was measured. The devised coating demonstrated login bacteria density (in CFU/cm²) of 1.36 ±0.18, whilst the control (without any coating) showed 3.42±0.09, which corresponds to log reduction of 2.06±0.21 (99.1% killed bacteria).

FIG. 3A A table demonstrating the long-term antibacterial activity of the devised coating formulation against Escherichia coli (E. coli). Each time point and log reduction data corresponds to the measured bacteria killing within 5 min after the bacteria have been introduced to the coating surface. *Log =1 means 1/10 survived (90% killed), 2 means 1/100 survived (99% killed), 3 means 1/1000 survived (99.9% killed) etc.

FIG. 3B is a graph demonstrating the long-term antibacterial activity of the devised coating formulation against Escherichia coli (E. coli). Each time point and log reduction data correspond to the measured bacteria killing within 5 min after the bacteria have been introduced to the coating surface.

FIG. 4 Photos demonstrating the stability of our coating formulation (ethyl cellulose (1.0 g, 300 cP), benzethonium chloride (0.2 g), benzalkonium chloride (0.1 g), and thymol (0.1 g) was dissolved in 100 ml of ethanol SDA 3C (95.2% ethanol, 4.8% isopropanol v/v), formulated as described in Example 1), and some commercial products (Complete Home™, Detrapel®, Microban®, and MonoFoil®) after manipulating the coated surface (polystyrene plastics) by 50 times finger swipes and 20 seconds rinsing with water.

FIG. 5 Photo demonstrating the friction stability of our coating formulation after manipulating the coated polystyrene plastic surface (1 ml solution of the coating formulation was sprayed by a commercial airbrush from an about 15 cm distance, as described in Example 2) by tape peeling (showed severe damage), fingernail scratch 240 times (showed severe damage), 1,200 times cloth wipes (showed slight damage), 1,200 times finger wipes (showed slight damage).

FIG. 6A Photo demonstrating the temperature stability of the coating formulation after coating a polystyrene plastic surface and then storing the coated materials at 60° C. for 24 h. Demonstrating a temperature stable coating with no damage.

FIG. 6B Photo demonstrating the temperature stability of the coating formulation after coating a polystyrene plastic surface and then storing the coated materials at —20° C. for 24 h. Demonstrating a temperature stable coating with no damage.

FIG. 7A Photo demonstrating the temperature storage stability of the coating solution after mixing the ingredients and then storing the solution at 60° C. for 24 h. Demonstrating a temperature stable solution without any precipitation or heterogeneity.

FIG. 7B Photo demonstrating the temperature storage stability of the coating solution after mixing the ingredients and then storing the solution at −20° C. for 24 h. Demonstrating a temperature stable solution without any precipitation or heterogeneity.

FIG. 8 Photo demonstrating the long-term storage stability of the coating solution after mixing the ingredients and then storing the solution at room temperature (r.t.) for 33 days. The solution remains colorless and without any precipitation or heterogeneity.

FIG. 9A Photo demonstrating the coating solution's stability against centrifugation. After centrifugation of the solution at 20,000 rcf for 1 h, the solution remained colorless and without any precipitation.

FIG. 9B Photo demonstrating the Ultraviolet (UV)-light stability of the coating formulation after coating a polystyrene plastic surface and then storing under UV-irradiation in a biosafety cabinet for 1 h. The experiments demonstrated UV-stable coating surfaces.

FIG. 9C Photo demonstrating the stability of the coating formulation after coating a polystyrene plastic surface and spitting on and wiping the surface. No damage to the surface was observed after the experiment.

FIG. 9D Photo demonstrating the stability of the coating formulation after coating a polystyrene plastic surface and spraying the commercial Lysol® All Purpose Cleaner onto the surface, followed by wiping the surface. Some damage to the surface was observed after the experiment.

FIG. 10 Photo demonstrating the transparency of the coated surface by visualizing the text completely when covered with the coated material. For example, “Coronavirus Disease 2019”, “CDC is responding to the novel coronavirus outb . . . ”, and “Learn More About COVID-19” are beneath the coated material and can be seen almost as clearly as the part uncovered/uncoated, such as “Is it COVID-19 or” and “Sepsis”.

FIG. 11 The first photo shows the finger rub. The other photos show the impact of finger rubs on the coating, with the number at the top left corner being the number of rubs. The surface was made of polystyrene and sprayed with an amount of 1 L per 50 m². The coating was intact until at least 500 rubs.

FIG. 12. The first photo shows the wiping with water. The other photos show the impact of such wiping on the coating, with the number at the top left corner being the number of wipes. The surface was made of polystyrene and sprayed with an amount of 1 L per 50 m². The coating was intact until at least 60 wipes.

FIG. 13. The first photo shows the wiping with water. The other photos show the impact of such wiping on the coating, with the number at the top left corner being the number of wipes.

The surface was made of polystyrene and sprayed with an amount of 1 L per 10.91 m². The coating was intact until at least 60 wipes.

FIG. 14. The photo shows the solution not centrifuged and after being centrifuged for 10 min at different speeds (100, 500, 10000, and 20000 rcf). No precipitate was observed for the 100 rcf one, and precipitates were observed for the 500, 10000, and 20000 rcf ones.

FIG. 15. The photos show the E. coli cultured after exposure to the coating formulation described in Example 1 (coated 0 days, 7 days, 15 days, and 31 days before the antibacterial experiment, formed by spraying 1 L per 50 m²) for 10 min (the “Our coating” row), and exposure to uncoated polystyrene surfaces (the “Control” row). Both experiments were repeated three times. On the Petri dishes, the numbers (0, −1, . . . , −5) at the tips of the pies indicate serial dilution to different bacteria concentrations relative to the original concentration (10°, 10⁻⁴, . . . , 10⁻⁵. The numbers on the edge of the pies indicate the number of colonies. Using the 0 day(s) (d) one as an example: For our coating, there were 0, 3, and 0 colonies at the original concentration. For controls, there were 5, 3, and 17 colonies at 100×dilution. Therefore, the percent of bacteria killed was 1−(0+3+0)/((5+3+17)*100)=99.88%. The percent of E. coli killed was 98.5%, 97.5%, and 60% for 7 days, 15 days, and 31 days, respectively.

### Procedure:

1. The bacteria were introduced to the coating as a suspension in water.
2. After collecting the bacteria by swabbing, the swabs were dipped into Dey-Engley Neutralizing Broth, and the serial dilutions were also using Dey-Engley Neutralizing Broth as the diluent.

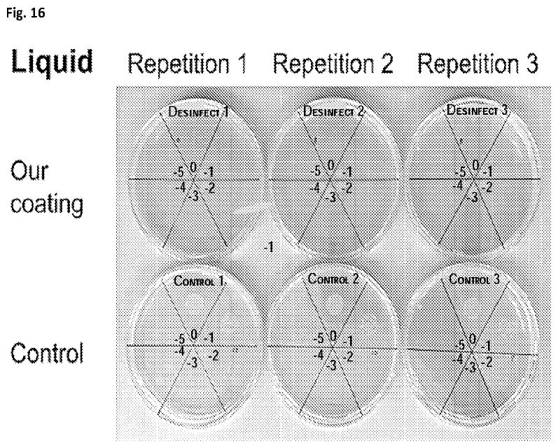

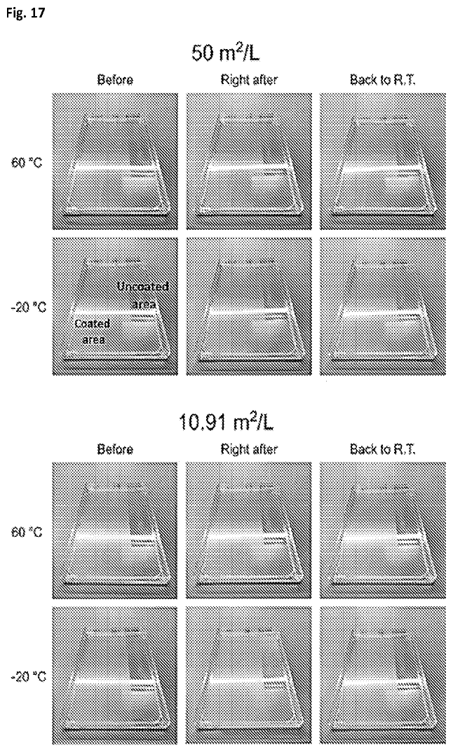

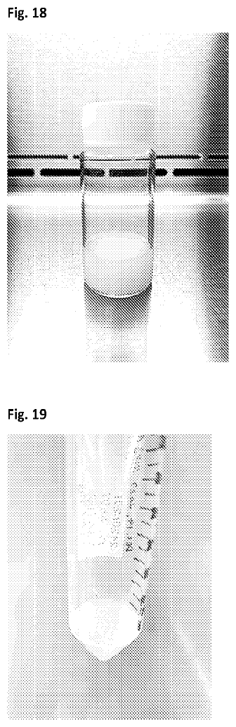

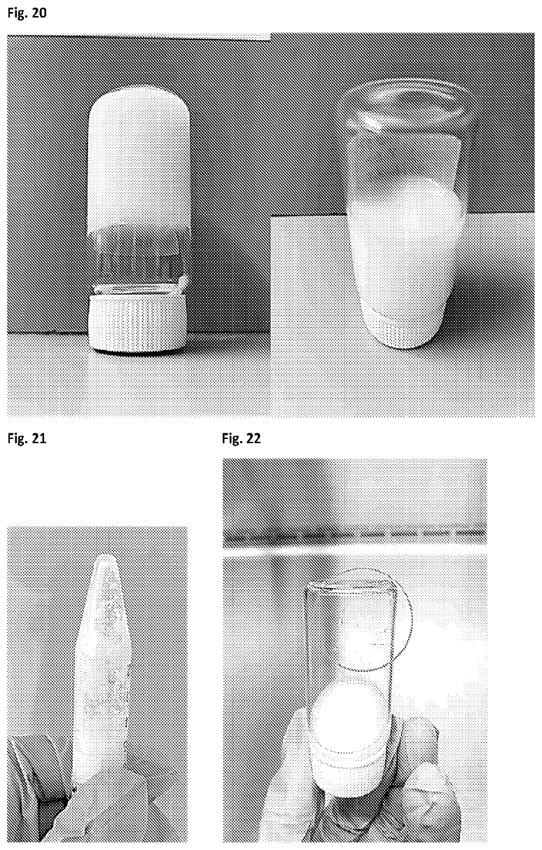

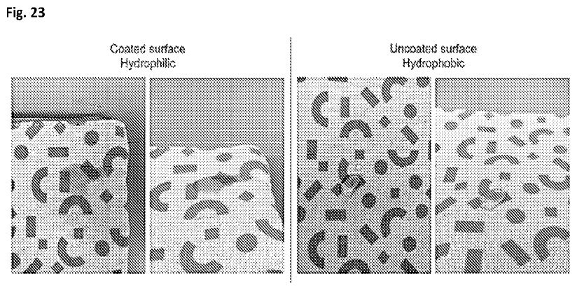

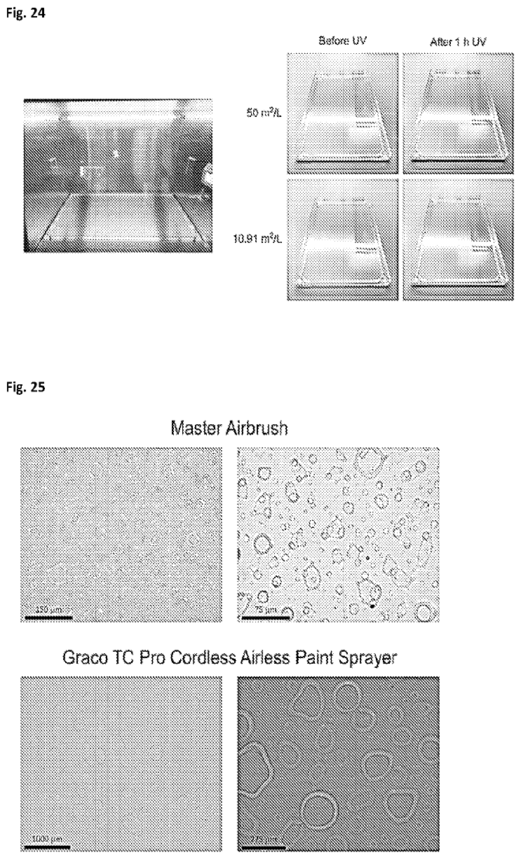

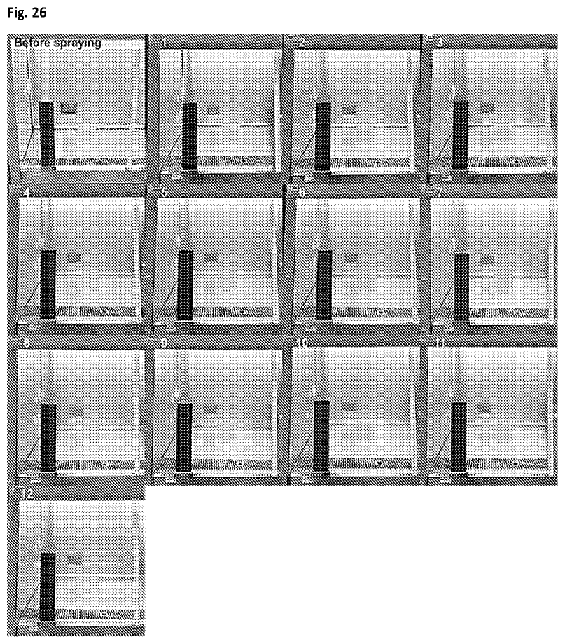

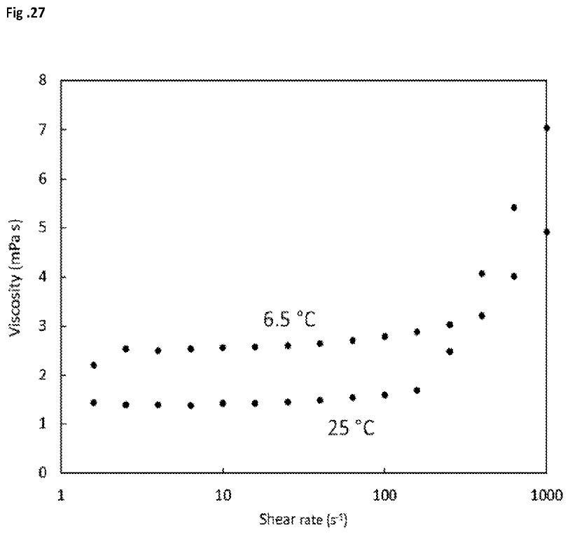

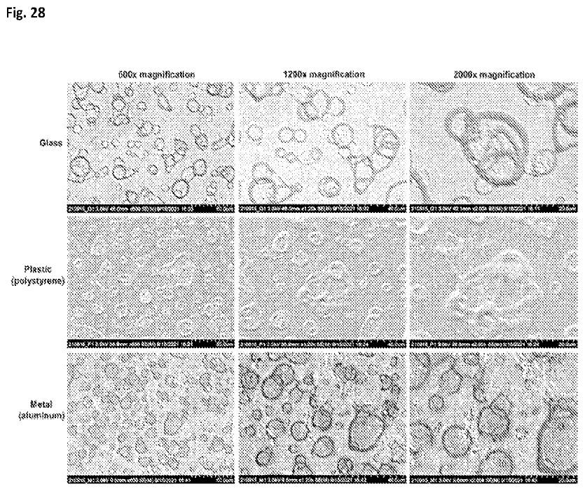

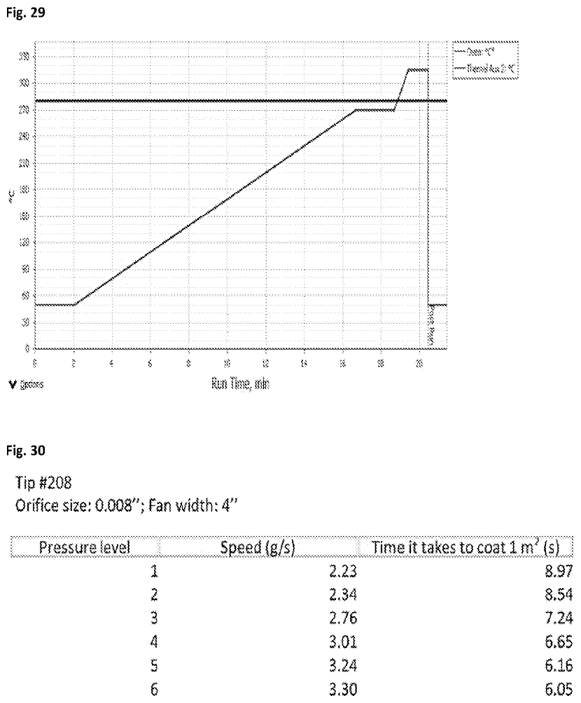

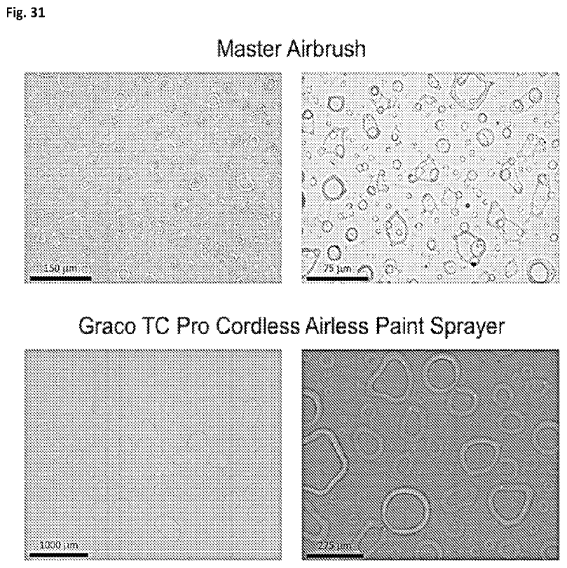

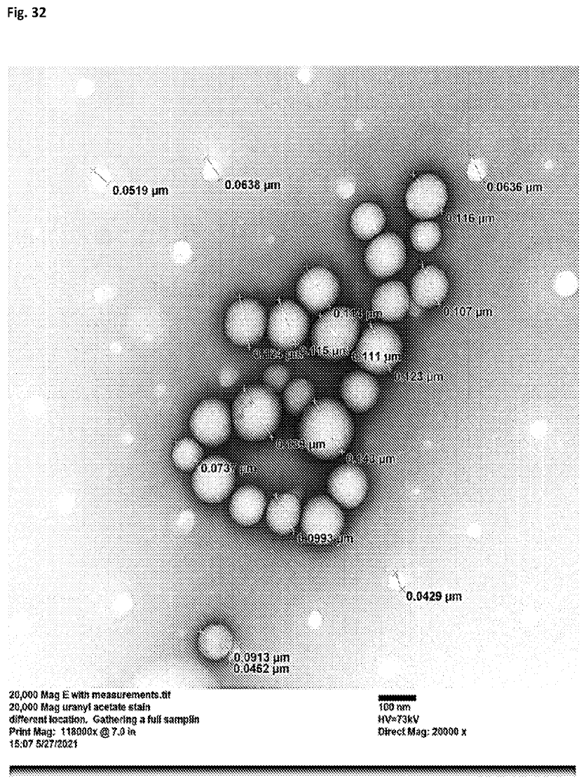

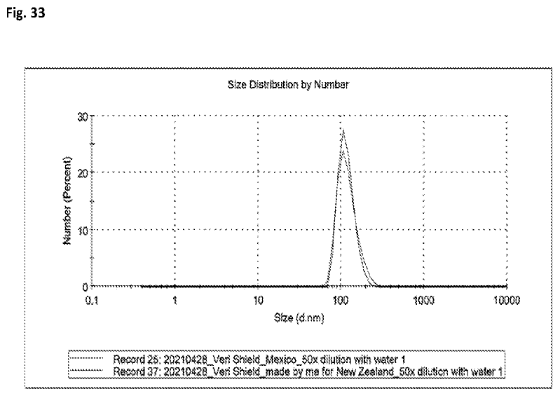

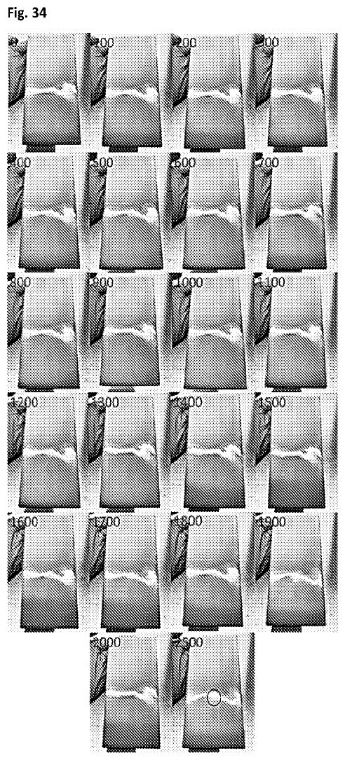

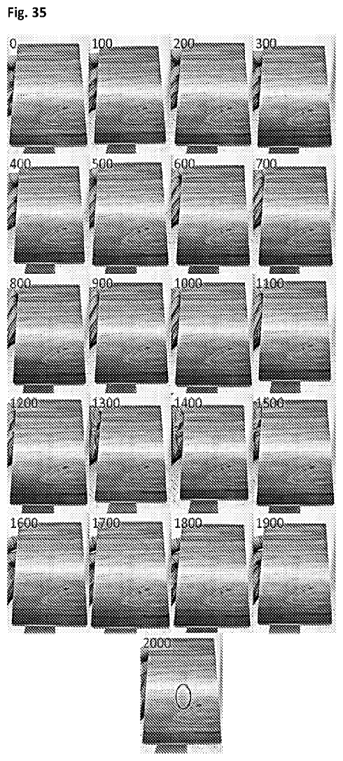

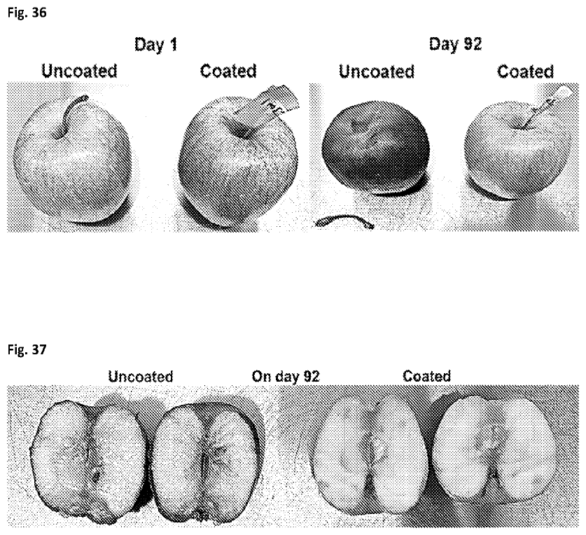

FIG. 16. The same volume of E. coli suspension was introduced onto the surface by swabbing as in FIG. 15. Then, the same amount of the coating formulation solution as described in Example 1 (liquid) was sprayed onto the surface as in FIG. 15. After 30 seconds, the bacteria were collected by swabbing, serially diluted, and cultured in the same way as in FIG. 15. The results show that all E. coli was killed, indicating efficacy of >99.97%.

FIG. 17. The photos show coatings with different sprayed amounts (50 m2/L and 10.91 m^{2/}L) before and right after being placed at 60° C. or −20° C. for 24 h, and when they were back to room temperature (R.T.).

FIG. 18. The the coating formulation solution as described in Example 1 displayed no visual changes after sitting at room temperature for 28 days.

FIG. 190. The coating formulation solution described in Example 1 displayed no visual changes after sitting at 4° C. for 16 days.

FIG. 20. Left: The the coating solution formulation described in Example 1 froze at −20° C.

Right: No phase separation was observed after it melts.

FIG. 21. Coagulation occurred after freezing the solution at −20 ° C., thawing, repeating this freeze-thaw cycle twice, freezing at −80° C., thawing. The melting point was measured to be −11° C. during the last thawing.

FIG. 22. The coating solution formulation described in Example 1 was contained in a vial, after sitting at 60° C. for 24 h. A slight number of solid pieces was formed (highlighted in the circle).

FIG. 23. The photos show water on a fabric surface coated (the two photos on the left) and not coated (the two photos on the right) with the coating formulation described in Example 1. The coated one had water absorbed into the fabric, and the uncoated one repelled the water. Therefore, the coated fabric was more hydrophilic than the uncoated one.

FIG. 24. Left: The coated substrates were placed at the center of a Labconco Purifier Biological Safety Cabinet for 1 h UV irradiation. One of the coatings was sprayed at an amount of 1 L per 50 m², the other 1 L per 10.91 m². Right: Neither of them displayed any visual changes after the UV irradiation.

FIG. 25. Optical microscope photos of the coatings (formulation described in Example 1). Top two photos: Sprayed by Master Airbrush (Multi-purpose Gravity Feed Dual-action Airbrush Kit with 6 Foot Hose and a Powerful ⅕ hp Single Piston Quiet Air Compressor) with an amount of 50 m²/L. Bottom two photos: Sprayed by Graco TC Pro Cordless Airless Paint Sprayer with a TC Pro 208 0.008 in. Tip at the pressure level 4 from a distance of about 1.5 m.

FIG. 26 Photo demonstrating the effect from the number of layers coated. The transparent surface turned opaquer with more layers of coating applied. The numbers at the top left corners of the photos are the number of layers coated. The spraying coverage was 50 m²/L for each layer.

FIG. 27 Graph demonstrating the viscosity the coating solution at 25° C. and 6.5° C. as a function of shear rate.

FIG. 28 Photo demonstrating the SEM images of the coated formulations on glass, plastic (polystyrene) and metal (aluminum) with 600×, 1200× and 2000× magnification.

FIG. 29. Temperature program in table 8.

FIG. 30. Demonstrate the time it takes for the Graco TC Pro SPRAYER to coat 1 m² of surface at 50 m²/L at different pressure levels.

FIG. 31 Photo demonstrating the optical microscope photos of the coatings. Top two photos: Sprayed by Master Airbrush (Multi-purpose Gravity Feed Dual-action Airbrush Kit with 6 Foot Hose and a Powerful ⅕ hp Single Piston Quiet Air Compressor) with an amount of 50 m^{2/}L. Bottom two photos: Sprayed by Graco TC Pro Cordless Airless Paint Sprayer with a TC Pro 208 0.008 in. Tip at the pressure level 4 from a distance of about 1.5 m.

FIG. 32 Photo demonstrating the transmission electron microscopy (TEM) image of coating solution formulation showing that it contains nanoparticles.

FIG. 33 Graph demonstrating the particle size distribution by number of the coating solution made in the lab (lower line) and the one made by the Mexican manufacturer (large scale production, higher line) determined by dynamic light scattering (DLS). The former has a Z-average diameter of 148.9±0.9 nm and a PDI of 0.050±0.014. The latter has a Z-average diameter of 134.7±1.0 nm and a PDI of 0.016±0.011. The two were very close, indicating that the manufacturer was producing a product consistent with what we made in the lab.

FIG. 34 The first photo shows the coated ceramic surface prior finger rub. The other photos show the impact of finger rubs on the coating, with the numbers of rubs are shown at the top left corner of the photos. The black circle highlights the damage done to the coating at the end.

FIG. 35 The first photo shows the coated wood surface prior finger rub. The other photos show the impact of finger rubs on the coating, with the numbers of rubs are shown at the top left corner of the photos. The red circle highlights the damage done to the coating at the end.

FIG. 36 The first photo shows the uncoated and coated apple at day 1. The second photo after day 92.

FIG. 37 The first photo shows the uncoated apple sliced in the middle at day 92. The second photo the coated apple sliced in the middle at day 92.

### DETAILED DESCRIPTION

### Definitions

As used herein “antimicrobial” means an agent or compound having the ability to kill or defect one or more bacteria, viruses, fungi and/or parasites. The antimicrobial may have antibacterial, antiviral, antifungal and antiparasitic disinfectant effects. The expression “one or more antimicrobial compounds” includes polymers thereof.

As used herein “disorder” includes any disease or illness or syndrome.

Kollicoat MAE 30 DP, CAS Number: CAS nr 25212-88-8, is a copolymer derived from methacrylic acid/ethyl acryliate, The ratio of the components in the copolymer are 1:1 and the average molecular weight is 250000 M/g. Kollicoat MAE 30 DP contains 0.7 wt % sodium lauryl sulfate and 2.3 wt % polysorbate 80 as surfactant/emylsifier/detergent.

Benzethonium chloride has structural formula

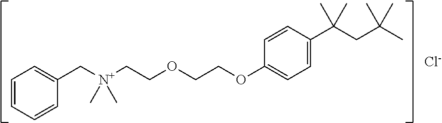

and systematic IUPAC name, Benzyldimethyl(2-{2-[4-(2,4,4-trimethylpentan-2-yl)phenoxy]ethoxy}ethyl)azanium chloride, and CAS Number 121-54-0.

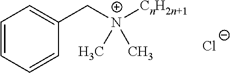

Benzalkonium chloride has structural formula n=8, 10, 12, 14, 16, 18 and IUPAC name N-Alkyl-N-benzyl-N,N-dimethylammonium chloride, and CAS Number 8001-54-5.

Thymol has structural formula

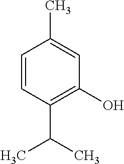

and systematic IUPAC name 5-Methyl-2-(propan-2-yl)benzenol and CAS Number 89-83-8.

The invention relates to a water insoluble film forming antimicrobial, antibacterial, antiviral, antiparasitic and antifungal composition, comprising or consisting of,

a) 0.1 to 10.0 wt %, or 1 to 6 wt % or 1 to 4 wt % of a film forming materials selected from the group comprising or consisting of ethyl cellulose, polyvinyl alcohol, polyvinyl acetate, polyvinyl alcohol-polyethylene glycol graft-copolymer, and/or an acrylate polymers, poly(methacrylic acid)polymer or co-polymers,

b) 0.15 to 0.80 wt %, or 0.20 to 0.60 wt % 0.40 to 0.60 wt % of one or more antimicrobial, antibacterial, antiviral, antiparasitic and antifungal compounds selected from the group comprising or consisting of thymol, benzethonium chloride, benzalkonium chloride, citric acid, lactic acid, carvacrol, 2-phenylphenol, chloroxylenol, 1,2-hexanediol, 1,2-octanediol, terpineol and 1,2-decanediol,

c) up to 100 wt % of one or more solubilizing agents, which is a solvent, or a mixture of solvents selected from the group comprising or consisting of water and a low molecular weight alcohol, such as ethanol and propanol, and

d) 0 wt % to 5 wt %, or 0.1 wt % to 1 wt %, one or more plasticizer,

e) optionally, one or more surfactants or detergents,

f) optionally, one or more agent selected from antivirals, antibacterial, antiparasitic, antifungal drug, mineral, vitamin, fatty acid, protein, odour masking agent, anti-itching agent, anti-inflammatory agent, and insecticidal compounds.

The invention relates to a water insoluble film forming antimicrobial, antibacterial, antiviral, antiparasitic and antifungal composition, comprising or consisting of,

a) 0.1 to 10.0 wt %, or 1 to 6 wt %, or 2 to 4 wt % of a film forming materials selected from the group comprising or consisting of an acrylate polymers or poly(methacrylic acid)polymer or co-polymers,

b) 0.15 to 0.80 wt %, or 0.20 to 0.60 wt %, or 0.40 to 0.60 wt % of one or more antimicrobial, 5 antibacterial, antiviral, antiparasitic and antifungal compounds selected from the group comprising or consisting of thymol, benzethonium chloride, benzalkonium chloride, 1,2-hexanediol, 1,2-octanediol, terpineol and 1,2-decanediol,

c) up to 100wt % of one or more solubilizing agents, which is a solvent, or a mixture of solvents selected from the group comprising or consisting of water and a low molecular weight alcohol, such as ethanol and propanol, and

d) 0 wt % to 5 wt %, or 0.1 wt % to 1 wt %, one or more plasticizer,

e) optionally, one or more surfactants or detergents,

f) optionally, 0 wt % to 25 wt %, or 10 wt % to 10 wt %, 0 wt % to 5 wt % of one or more agent selected from antivirals, antibacterial, antiparasitic, antifungal drug, mineral, vitamin, fatty acid, protein, odour masking agent, anti-itching agent, anti-inflammatory agent, and insecticidal compounds.

The acrylate polymers or co-polymers may be selected from the group comprising or consisting of methacrylic acid-ethyl acrylate copolymer and methacrylic acid-acrylic ester copolymer. The acrylate polymers or co-polymers may be methacrylic acid-ethyl acrylate copolymer or Kollicoat MAE 30 DP™.

The amount of film forming materials, or methacrylic acid-ethyl acrylate copolymer or Kollicoat MAE 30 DP™ in the final composition may be 0.5 to 20 wt %, or 1 to 15 wt %, or 1 to 5 wt %, 1.5 to 5 wt %, or 2 to 4 wt %, or 2.5 to 3.5 wt %.

The one or more antimicrobial, antibacterial, antiviral, antiparasitic and antifungal compounds is selected from the group comprising or consisting of thymol, carvacrol, 1,2-hexanediol and 1,2-octanediol. The one or more antimicrobial, antibacterial, antiviral, antiparasitic and antifungal compounds is thymol and/or 1,2-hexanediol.

The amount of the antimicrobial, antibacterial, antiviral, antiparasitic and antifungal compound may be 0.01-0.8 v/v % of 1,2-hexanediol and 0.01-0.8 w/v % of thymol, or 0.1-0.5 v/v % of 1,2-hexanediol and 0.1-0.5 w/v % of thymol, or 0.1-0.3 v/v % of 1,2-hexanediol and 0.1-0.3 w/v % of thymol, or 0.25-3.2 v/v % 1,2-hexanediol and 0.15-0.25 w/v % thymol.

The amount of the antimicrobial, antibacterial, antiviral, antiparasitic and antifungal compound may be 0.30 v/v % 1,2-hexanediol and 0.24 w/v % thymol.

The solvent may be a mixture of ethanol and isopropanol. The solvent may be a mixture of water and ethanol. The amount of ethanol in a mixture of water and ethanol solvent based on the weight of the final composition may be 5 to 50 wt %, or 5 to 25 wt %, or 5 to 20 wt %, 5 to 15 wt %, or 10 to 20 wt %, or 10 to 15 wt %.

The plasticizer may be selected from the group comprising or consisting of glyceryl trioctanoate, dibutyl sebacate, triethyl citrate, triacetin, Myvacet® (acetylated monoglycerides) and diethyl phthalate. The plasticizer may be glyceryl trioctanoate.

The amount of glyceryl trioctanoate present may be from 0.01 wt % to 20 wt %, or 0.01 wt % to 10 wt %, or 0.05 wt % to 5 wt %, or 0.1 wt % to 1 wt %, or 0.01 wt % to 1 wt %, or about 0.1 wt %. Percentages based on the weight of the final composition

The surfactant or detergents or a mixture of surfactants or detergents may be polysorbate 80 and/or sodium lauryl sulfate.

The water insoluble film forming antimicrobial, antibacterial, antiviral, antiparasitic and antifungal composition may comprise or consists of
a) 1 to 5 wt % a film forming materials selected from Kollicoat MAE 30 DP™,
b) 0.30 to 0.75 one or more antimicrobial, antibacterial, antiviral, antiparasitic and antifungal compounds selected from the group comprising or consisting of thymol and 1,2-hexanediol,
c) up to 100 wt % one or more solubilizing agents selected from water and ethanol, whereby the weight ratio ethanol to water is 5-20 to 95 to 80.

In some aspects, the water insoluble film forming antimicrobial, antibacterial, antiviral, antiparasitic and antifungal composition further comprises one or more odour masking agent, optionally selected from the group comprising or consisting of menthol, citronellal, citrus, lemon, fragrances, such as Fragrance Lemon Verbena (Making Cosmetics Lot # 071118MCLV) (preferable concentrations 0.05%, 0.1%, 0.2%, 0.5%, and 1%, or 0.1% or 0.2%), Fragrance Blood Orange (Making Cosmetics Lot #171101015), Fragrance Pink Grapefruit Passion Fruit (Making Cosmetics Lot #180728016), Fragrance Mangosteen (Making Cosmetics Lot #180620050), Fragrance Coral Reef (Making Cosmetics Lot #170815034), Fragrance Citrus Punch (Making Cosmetics Lot #180206062), lemon fragance, and orange fragance and lavender fragrance.

In some aspects, the water insoluble film forming antimicrobial, antibacterial, antiviral, antiparasitic and antifungal composition further comprises one or more anti itching agent, optionally selected from the group comprising or consisting of pramoxine hydrochloride, diphenhydramine, cetirizine, loratadine, desloratadine and fexofenadine.

In some aspects, water insoluble film forming antimicrobial, antibacterial, antiviral, antiparasitic and antifungal composition further comprises or consists of one or more anti-inflammatory agent, optionally selected from the group comprising ibuprofen, naproxen, diclofenac, celecoxib, mefenamic acid, etoricoxib, indomethacin, aspirin, diflunisal and neosporin.

The composition of the invention may be used for coating animated and non-animated surfaces. The surfaces may be any organic surface, such as fruits, vegetables, nuts, meat, beef, fish, bone, or any edible product. The surfaces may be foods, fruits and vegetables. The surfaces may be an apple or an orange.

The composition of the invention may be used as a pesticide, insecticide or ant-microbal agent to prevent microbial deposition, growth, spread and survival.

The composition of the invention may be used in therapy, such as for use in prevention or treatment of a disorder in a mammal, such as a human. The disorder may be a skin disorder.

In some aspects, treating or preventing occurs by an application method selected from the group consisting of spraying, pressurized aerosol, fogging, rolling, brushing, mopping, wiping, dipping, injecting or any mixture thereof.

In some aspects, the film forming antimicrobial, antibacterial, antiviral, antiparasitic and antifungal composition, the composition as defined herein can optionally be composed of a combination of zero, one or more of the antivirals, antibacterial, antiparasitic, antifungal drug, mineral, vitamin, fatty acid, protein, odour masking agent, anti-itching agent, anti-inflammatory agent, and insecticidal compound.

In certain aspects, the invention relates to an environmentally friendly antimicrobial and antiviral surface disinfectant and long-lasting coating formulation. In some aspects the composition comprises film forming material, antimicrobial, antiviral, biocidal and quaternary ammonium-based compounds and polymers, and combination thereof, solubilizing agents and optionally plasticizer. In some aspects the formulation may be used to treat or prevent microbial, fungal, parasitic deposition and viral formation, spreading and survival. In some aspects, the formulation may be used to immediately kill microbes and viruses and immediately form a long-lasting, transparent coating with the ability to further protect the surface for a long period of time. In some aspects, the formulation may be used to treat any surfaces selected from the group comprising plastics, metal, steel, wood, ceramics, tissue, glass, granite, stone, marble, hard and soft surfaces, skin or mucosal surface. In some aspects the formulation may be used to treat surfaces of fruits, vegetables, nuts, meat, fish, bone or any edible products, or any mixture thereof, any type of trees, plants, flowers, crops, harvest plats, retails and hospitals, public spaces, hotels, boats, airports, packages, boxes, bin, toilets, bathrooms, domestic and professional settings, and combination thereof. In some aspects, the formulation may be administered or applied, for instance by spraying, pressurized aerosol, fogging, rolling, brushing, mopping, wiping and dipping.

In certain aspects, the invention relates to a composition comprising film forming material selected from cellulose or its derivatives, antimicrobial, antiviral and biocidal components, and combination thereof, and solubilizing agents.

In certain aspect, the invention relates to a composition comprising film forming material selected from cellulose or its derivatives, physically entrapped or covalently functionalized with antimicrobial, antiviral, biocidal and quaternary ammonium-based compounds and polymers, and combination thereof, and solubilizing agents.

In some aspects the composition comprises film forming material ethyl cellulose, the antimicrobial and antiviral comprises a combination of benzethonium chloride, benzalkonium chloride and thymol, and the solubilizing agent ethanol.

In certain aspects, the formulation comprises a combination of aqueous media and an organic liquid, or solvent mixture of any low molecular weight alcohols, water, alkylene glycol ether based, oils, buffers organic solvent, and combination thereof.

Ethyl cellulose is a biocompatible and Food and Drug Administration (FDA)-approved polymer. When combined with appropriate solvent forms a stable film immediately after applied.

In some aspects the composition comprises Environmental Protection Agency (EPA)-accepted ingredients.

In certain aspects, the invention relates to antibiotic free agents. In one aspect, the use of antibiotics is disclaimed.

In certain aspects, the formulation immediately upon application form a stable, transparent and long-lasting coating.

In certain aspects, the composition, upon application, forms a stable and transparent coating, thereby substantially killing, preventing and protecting from microbial, fungal, parasitic and viral formation, spreading and survival.

In certain aspects, the disclosure further provides medical and veterinary uses for the formulation. While human applications will become apparent from the disclosure, a preferred use relates to a method of forming an environmentally friendly antimicrobial and antiviral surface disinfectant and long-lasting coating formulation protecting various surfaces.

In certain aspects, the surface may be made from selected from the group comprising to organic and inorganic materials, plastics, metal, steel, wood, ceramics, tissue, glass, granite, stone, marble, hard and soft surfaces, skin or mucosal surface.

In certain aspects, the surface may selected from the group comprising fruits, vegetables, retails and hospitals, public spaces, hotels, boats, airports, packages, boxes, bins, toilets, edible products, bathrooms, domestic and professional settings.

In particular aspects, the formulation may be administered or applied by spraying.

### EXAMPLES

In order that aspects of the invention described herein may be more fully understood, the following examples are set forth. The examples described in this application are offered to illustrate the compounds, pharmaceutical compositions, and methods provided herein and are not to be construed in any way as limiting their scope.

### Materials and Methods

Chemicals and solvents were purchased from commercial suppliers or were fabricated and purified by standard techniques. The following commercial reagents were used as purchased without any further purification: ethanol SDA 3C (USA LAB ALLEY, EAS1014-55GAL, USA), ethyl cellulose (Viscosity 300 cP, 200654, Sigma Aldrich), benzethonium chloride (≥98% (HPLC), Sigma Aldrich, B8879), benzalkonium chloride (41339, Alfa Aesar) and thymol (Crstl, NF, 6WYW8, GRAINGER).

### Example 1—Preparation of the Coating Solution

The various compositions ethyl cellulose (300 cP, 1.0 g), benzethonium chloride (0.2 g), benzalkonium chloride (0.1 g) and thymol (0.1 g) was dissolved in 100 ml of ethanol SDA 3C (95.2% ethanol, 4.8% isopropanol v/v) by stirring at room temperature until a homogenous solution was formed.

### Example 2—Film Preparation of the Formulation on Various Surfaces

The film coating on various surfaces such as polystyrene plastics, metal, wood, and ceramics was performed using a commercial airbrush (Master airbrush multi-purpose gravity feed dual-action airbrush kit with 6-foot hose and a powerful ⅕ HP single piston quiet air compressor). The coating formulation solution (1 ml) was pipetted into the gravity fluid cup of the airbrush and subsequently the entire solution was sprayed on the selected surface from a about 15 cm distance.

Example 3—Evaluation of the Antimicrobial Properties of the Disinfectant Formulation

The bacteria strains Staphylococcus aureus (S. aureus) and Escherichia coli (E. coli) strains were used in all the experiments. Prior to each experiment, bacterial stock cultures were prepared by inoculating a single bacterial colony in 3.7% Brain Heart Infusion (BHI) broth at 37° C. until stationary phase. Bacterial stock cultures were then diluted to 5×10⁸ colony forming units (CFU)/ml. To test the antibacterial properties of disinfectant solution, about 10⁷ CFU of the selected bacteria was added to a plate lid surface (127.7 mm×85.4 mm) by taking a dip swap from the bacteria suspension and then further introducing the bacteria to the surface (about 10⁵ CFU/cm²), subsequently 1 ml of the formulation was sprayed on this surface. The antibacterial activity was measured immediately and after 10 min by collecting the bacteria by taking a swab from the surface and then further suspend the bacteria in PBS and further bacteria culturing to quantify the level of bacteria presence after incubating the agar plates for 16 h. Bacterial density on each sample (CFU/cm²) was determined by the number of colonies counted. The control sample contained only bacteria solution on the surface without any treatment of the disinfectant solution. The coating showed a bacteria density of 3.43±0.06 and the control 4.05±0.21 (CFU/cm²), which corresponds to 0.63±0.22 log reduction or 76% killing after immediate measurements. After 10 min of incubation and then further measurements of the coating showed a bacteria density of 1.81±0.10 and the control 4.03±0.15 (CFU/cm²), which corresponds to 2.21±0.18 log reduction or 99.4% killing.

### Example 4—Evaluation of the Antimicrobial Properties of the Coated Formulation

The antimicrobial formulation (1 ml) was sprayed onto a plate lid surface (polystyrene plastic), which immediately formed a stable and transparent film. To test the antibacterial properties of coated surface, a solution of 5×10⁸ CFU/ml of the E. coli was added to a plate lid surface (127.7 mm×85.4 mm) by taking a dip swap from the bacteria suspension (about 10⁷ CFU) and then further introducing the bacteria to the surface (about10⁵ CFU/cm²).

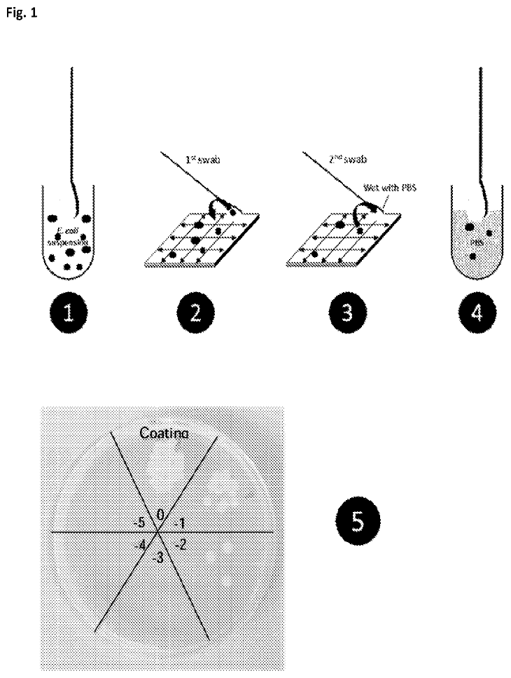

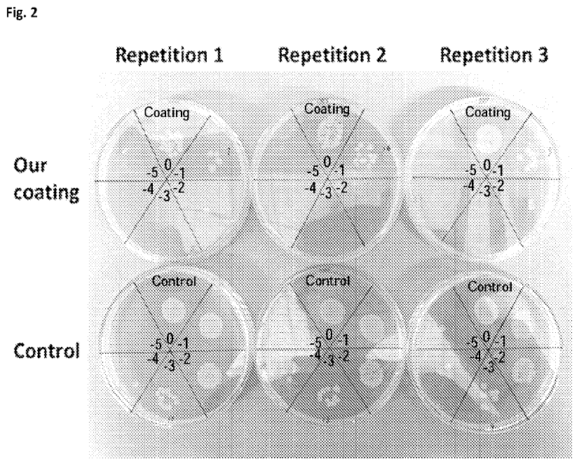

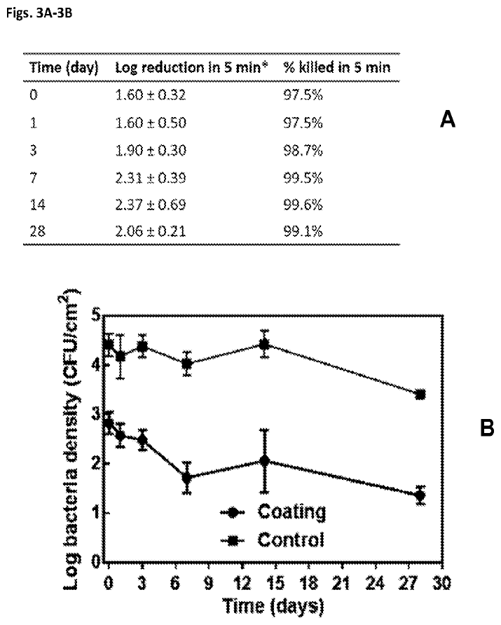

The antibacterial activity was measured on the selected time points by collecting the bacteria by taking a swab from the surface and then further suspend the bacteria in PBS and further bacteria culturing to quantify the level of bacteria presence by incubating the agar plates for 16 h. Bacterial density on each sample (CFU/cm²) was determined by the number of colonies counted. The control sample contained only bacteria solution on the surface without any treatment of the disinfectant solution. See FIGS. 1, 2, 3A and B.

### Example 5—Stability Evaluation of our Coating Formulation and Commercial Products

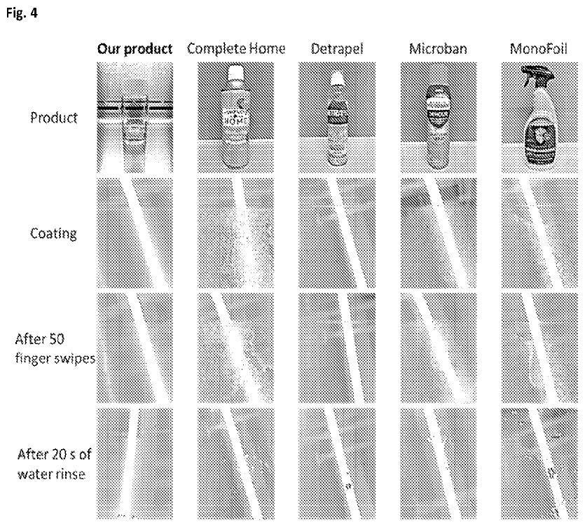

The stability of the coating formulation solution and its corresponding coated surface film was investigated through various experiments. See FIGS. 4-9.

Experiment 1: The stability of our coating formulation and some commercial products (Complete Home™, Detrapel®, Microban®, and MonoFoil®) was investigated by spraying the respective products onto a polystyrene plastic surface, followed by manipulating the coated surface by 50 times finger swipes and 20 seconds rinsing with water. The coated film showed no damage, whilst the commercial products were completely destroyed, except for the coating from the Detrapel®. See FIG. 4.

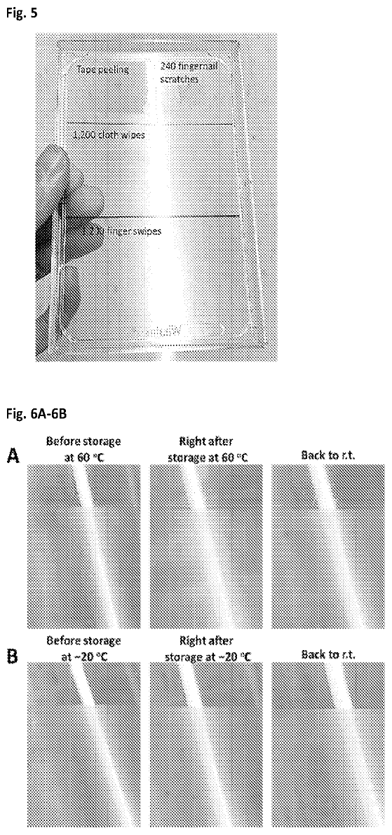

Experiment 2: The friction stability of our coating formulation was investigated by spraying the devised product onto a polystyrene plastic surface (1 ml solution of the coating formulation was sprayed by a commercial airbrush from a about 15 cm distance, as described in Example 2), followed by manipulating the coated surface by tape peeling, fingernail scratch 240 times, 1,200 times cloth wipes and 1,200 times finger wipes. See FIG. 5.

Experiment 3: The temperature stability of the coating formulation was investigated by coating a polystyrene plastic surface and then storing the coated materials at 60° C. and —20 C. for 24 h. See FIGS. 6A and 6B.

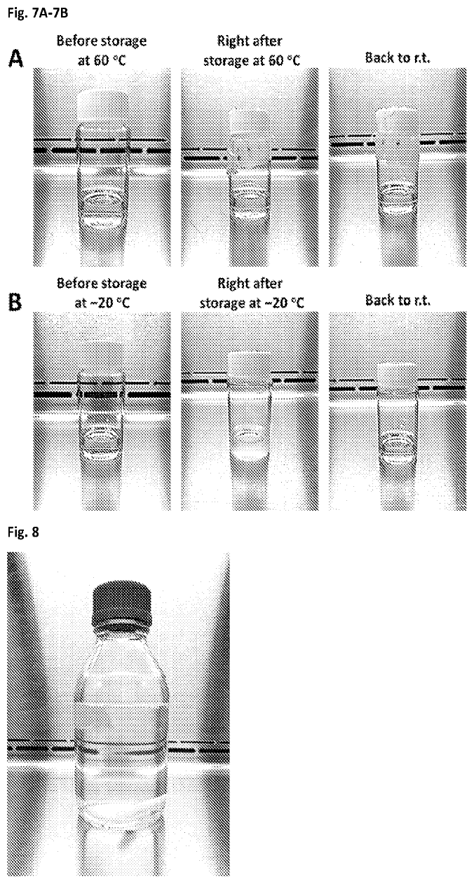

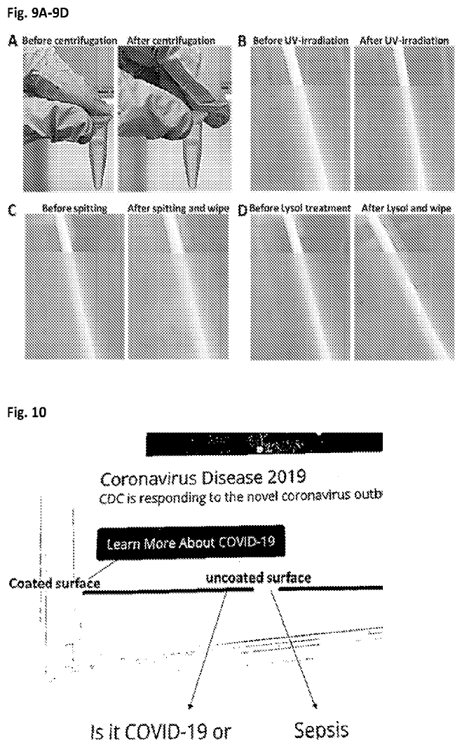

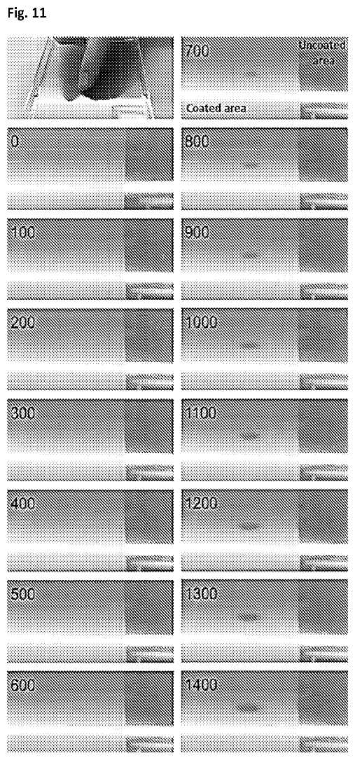

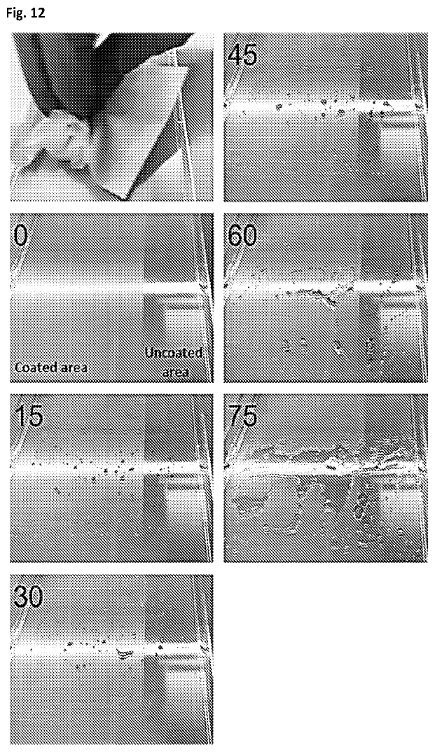

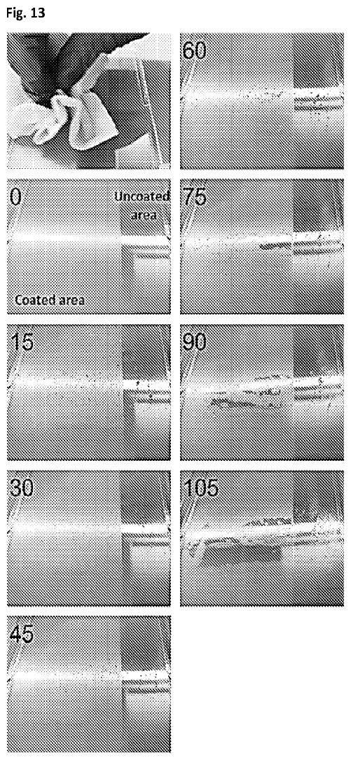

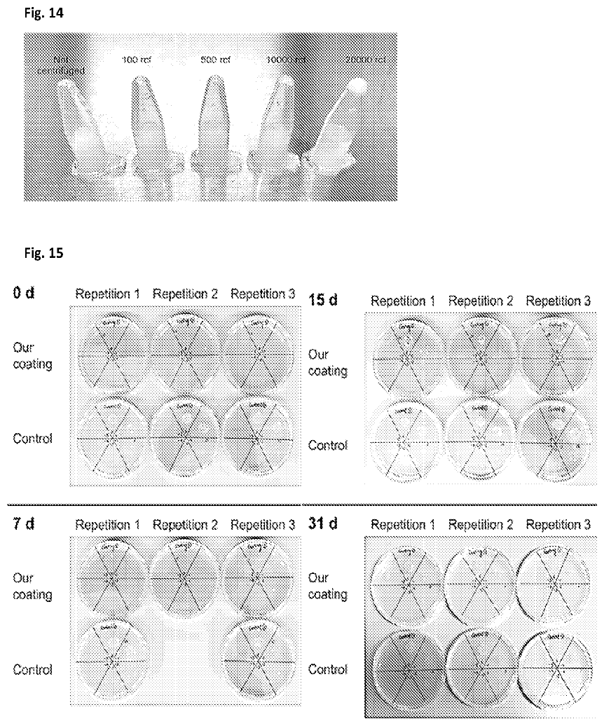

Experiment 4: The temperature storage stability of the coating solution was performed by mixing the ingredients and then the solution was further stored at 60° C. and −20° C. for −24 h. See FIGS. 7A and 7B.

### Experiment 5: The long-term storage stability of the coating solution was investigated by storing the solution at r.t. for 33 days. See FIG. 8.

### Experiment 6: The stability of the coating solution against centrifugation was investigated by centrifuging the solution at 20,000 rcf for 1 h. See FIG. 9A.

Experiment 7: The UV-light stability of the coating formulation after coating a polystyrene plastic surface was investigated by 254 nm UV-irradiation of the coated surface in a biosafety cabinet for 1 h. See FIG. 9B.

Experiment 8: The stability of the coating formulation after coating a polystyrene plastic surface was further investigated by spitting (about 2 ml) and wiping the coated surface. See FIG. 9C.

Experiment 9: The stability of the coating formulation after coating a polystyrene plastic surface was investigated by spraying the commercial Lysol®. All Purpose Cleaner disinfectant spray (about 0.5 ml) onto the surface, followed by wiping the surface. See FIG. 9D.

### Materials and Methods

Chemicals and solvents were purchased from commercial suppliers or were fabricated and purified by standard techniques. The following commercial reagents were used as purchased without any further purification: Ethanol (96%, Reag. Ph Eur, Sigma Aldrich), Kollicoat® MAE DP (46.0-50.6% methacrylic acid basis, Sigma Aldrich), thymol (Crstl, NF, 6WYW8, GRAINGER) and 1,2-hexanediol (98%, Sigma Aldrich).

### Example 6—Preparation of the Coating Solution

1. Dissolve 2.3627 g of thymol and 2.8087 g of 1,2-hexanediol in 122.73 g of 96 v/v % ethanol (the 4 v/v % is water) by stirring for 2 min.
2. Under stirring, add 807.67 g of water, continue to stir for 5 min to mix well.
3. Under stirring, add 32.815 g of Kollicoat® MAE 30 DP, continue to stir for 15 min to mix well.

### Additional Notes:

All steps are conducted at room temperature.

### Example 7—Evaluation of the Antimicrobial Properties of the Disinfectant Formulation

The bacteria strains Staphylococcus aureus (S. aureus) and Escherichia coli (E. coli) strains were used in all the experiments. Before each experiment, bacterial stock cultures were prepared by inoculating a single bacterial colony in 3.7% Brain Heart Infusion (BHI) broth at 37° C. until stationary phase. The bacteria suspension was centrifuged at 1000 rcf for 10 min at 20° C. to precipitate the bacteria. The supernatant (BHI broth) was decanted and replaced with the same volume of Milli-Q water. It was then vortexed to redisperse the bacteria in water. To test the antibacterial properties of the disinfectant solution, 1104 of the bacteria water dispersion was added to a plate lid surface (127.7 mm×85.4 mm) and spread out by swabbing with a Q-Tips cotton swab, subsequently, 0.218 ml (corresponding to 50 m^{2/}L) of the formulation was pipetted on this surface and spread out with another Q-Tips cotton swab. After 30 seconds, this swab was agitated in 500 μL of Dey-Engley neutralizing broth to detach the bacteria. After serial dilution and incubation on agar plates at 37° C. for 12-16 h, the bacterial density on each sample (CFU/cm²) was determined by the number of colonies counted. The control sample used Milli-Q water in place of the disinfectant solution.

### Example 8—Evaluation of the Antimicrobial Properties of the Coated Formulation

The antimicrobial formulation (0.218 ml, corresponding to 50 m^{2/}L) was sprayed onto a plate lid surface (polystyrene plastic, 127.7 mm x 85.4 mm) and allowed to dry for 3 min, which formed a stable and transparent film. To test the antibacterial properties of the coated surface, 1104 of a bacteria water dispersion (prepared in the same way as in Example 3) was added to a plate lid surface and spread out by swabbing with a Q-Tips cotton swab. After 10 minutes, another Q-Tips cotton swab fully soaked with water was used to swab and collect the bacteria on the surface. This swab was agitated in 500 μL of Dey-Engley neutralizing broth to detach the bacteria. After serial dilution and incubation on agar plates at 37° C. for 12-16 h, the bacterial density on each sample (CFU/cm²) was determined by the number of colonies counted. The control sample skipped the spraying and film formation steps.

### Example 9—Screening of the Coating Formulations with Various Antimicrobial Ingredients

After collecting the bacteria by swabbing, the swabs were dipped into PBS, and the serial dilutions were also using PBS as the diluent. All experiments were conucted right after the spraying of coating formulations and the subsequent formation of the coating. The various antimicrobial ingrenteksns and their combination screened are presented in Table 1.

TABLE 1
Screening of formulations for the highest antibacterial efficacy.

| Thymol | Second anti- | Second agent | E. coli killed |
|---|---|---|---|
| concentration^{[a]} | microbial agent | concentration^{[a]} | after 5 min |
| 0.20^{[b]} | Citric acid | 0.20 | 69% |
| 0.20^{[b]} | 1,2-Hexanediol | 0.20 | 98% |
| 0.20^{[b]} | Lactic acid | 0.20 | 46% |
| 0.20^{[b]} | Octanoic acid | 0.20 | 83% |
| 0.20^{[c]} | 1,2-Hexanediol | 0.15 | 87% |
| 0.25^{[c]} | 1,2-Hexanediol | 0.15 | 65% |
| 0.25^{[c]} | 1,2-Hexanediol | 0.20 | 85% |
| 0.20^{[c]} | 1,2-Hexanediol | 0.25 | 99.975% |
| 0.25^{[c]} | 1,2-Hexanediol | 0.25 | 99.915% |

^{[a]}Except for the active ingredients, all other ingredients and their concentrations were the same as the coating formulation presented in Example 1. Here, the concentrations are relative to the methacrylic acid-ethyl acrylate copolymer (1:1). It is w/w for solids and v/w for liquids.
^{[b]}The bacteria were introduced to the coating as a suspension in PBS.
^{[c]}The bacteria were introduced to the coating as a suspension in pure water.

TABLE 2
The time it takes for the Graco TC Pro Sprayer to coat 1 m²
of surface at 50 m²/L at different pressure levels
Tip #208
Orifice size: 0.008″; Fan width: 4″

| Pressure level | Speed (g/s) | Time it takes to coat 1 m² (s) |
|---|---|---|
| 1 | 2.23 | 8.97 |
| 2 | 2.34 | 8.54 |
| 3 | 2.76 | 7.24 |
| 4 | 3.01 | 6.65 |
| 5 | 3.24 | 6.16 |
| 6 | 3.30 | 6.05 |

### Motivation for the Selection of the Active Ingredients

All the antimicrobial active agents selected are active ingredients in the EPA's List N (Disinfectants for Use Against SARS-CoV-2 (COVID-19)).

Moreover, the 1,2-hexanediol not only acts as an active antimicrobial agent but also acts as a plasticizer and therefore promote the self-assembly of the polymers towards a more robust film coating. The use of thymol have also additional advantages beside being antimicrobial, it has been reported by Palaniappan et al. that thymol is highly effective in reducing the minimum inhibitory concentration of several antibiotics.¹ Liu et al. reported that thymol elicited a synergistic effect with streptomycin against L. monocytogenes.² Zhou et al. found that thymol has a synergistic effect with chelators and organic acids against Salmonella Typhimurium.³ The antimicrobial efficacy of soy sauce can be enhanced by thymol acting in synergism as reported by Moon et al.⁴ In expectation for synergy, thymol was added in our final formulation.

### Example 10—Antibacterial and Antifungal Tests

E. coil was grown in a BHI medium to a concentration of 10⁹ CFU/mL at 37° C. and 250 rpm shaking. For Candida albicans, it was grown in a Sabouraud dextrose broth to a concentration of 10⁸ CFU/mL at 30° C. and 250 rpm shaking (shaking was necessary to avoid dumping of the fungi). The liquid medium with bacteria or fungi was centrifuged at 1000 rcf (for bacteria) or 250 rcf (for fungi) for 20 min to precipitate the bacteria or fungi. The supernatant was carefully decanted, and the same volume of sterile pure water was added. It was then vortexed to resuspend the bacteria or fungi in water. This suspension should be used immediately. 110 μL of the suspension was pipetted to the surface and spread out by swabbing for 1 min. Here, a dry swab was used for bacteria; while for fungi, the swab was dipped into the fungi suspension to get it wet right before using it to spread. The Petri dishes were with the lids on throughout the 9 min (9 min+1 min of spreading time=10 min) waiting time for bacteria; while for fungi, the Petri dishes were with the lids on for 3 h 19 min, and remove the lids to let it dry for the remaining 40 min (4 h in total). After the waiting, all the Petri dishes (bacteria or fungi) would be dry or almost dry. 100 μL of water was pipetted onto each Petri dish, which was then swabbed using a dry and clean swab for 1 min to collect the bacteria or fungi. The swab was then dipped into 600 μL of Dey-Engley neutralizing broth and agitated up-and-down for 50 times to detach the bacteria or fungi from the swab. The broth was serially diluted to 10, 100, 1000, 10⁴, and 10⁵ times with also the Dey-Engley neutralizing broth. Including the undiluted one, 20 μL of each of the six was spotted on an agar plate. The plates were incubated at 37° C. until visible colonies could be seen and counted. It took 14 h for E. coil and 26 h for Candida albicans.

TABLE 3
E. coli killing efficacy of various formulas right after coating and drying with
a bacteria exposure time of 10 min. The spraying coverage was 50 m²/L (157 μL per
Petri dish). The antibacterial tests were conducted right after the coatings were
dried and formed. The bacteria were exposed to the coating for 10 min.

| | | | E. coli killing | Other |
|---|---|---|---|---|
| Alcoholics | Phenolics | Polymer | efficacy | observations |
| Hex (0.30%) | Thymol (0.24%) | Kollicoat MAE 30 | 99.43% | |
| | | DP (1%) | | |
| Hex (0.45%) | Thymol (0.36%) | Kollicoat MAE 30 | 99.79% | |
| | | DP (1%) | | |
| Hex (0.60%) | Thymol (0.48%) | Kollicoat MAE 30 | 87.14% | |
| | | DP (1%) | | |
| Hex (0.75%) | Thymol (0.60%) | Kollicoat MAE 30 | 94.55% | |
| | | DP (1%) | | |
| Hex (0.90%) | Thymol (0.72%) | Kollicoat MAE 30 | 91.82% | |
| | | DP (1%) | | |
| Hex (1.50%) | Thymol (1.20%) | Kollicoat MAE 30 | Not effective | |
| | | DP (1%) | | |
| Oct (0.30%) | Thymol (0.24%) | Kollicoat MAE 30 | 99.57% | |
| | | DP (1%) | | |
| Dec (0.30%) | Thymol (0.24%) | Kollicoat MAE 30 | Not effective | |
| | | DP (1%) | | |
| Hex (0.30%) | Thymol (0.24%) | Kollicoat SR 30 D | Not effective | |
| | | (1%) | | |
| Hex (0.30%), Ter | Thymol (0.24%) | Kollicoat MAE 30 | 76% | |
| (0.30%) | | DP (1%) | | |
| Oct (0.30%), Ter | Thymol (0.24%) | Kollicoat MAE 30 | 90% | Weird odor; |
| (0.30%) | | DP (1%) | | droplets join |
| | | | | each other and |
| | | | | form bigger |
| | | | | droplets |
| Hex (0.15%), Oct | Thymol (0.24%) | Kollicoat MAE 30 | 80% | |
| (0.15%), Ter | | DP (1%) | | |
| (0.30%) | | | | |
| Ter (0.30%) | Thymol (0.24%) | Kollicoat MAE 30 | 77% | |
| | | DP (1%) | | |
| Hex (0.30%), Ter | — | Kollicoat MAE 30 | 89% | |
| (0.30%) | | DP (1%) | | |
| Oct (0.30%), Ter | — | Kollicoat MAE 30 | 77% | Weird odor; |
| (0.30%) | | DP (1%) | | droplets join |
| | | | | each other and |
| | | | | form bigger |
| | | | | droplets |
| Ter (0.30%) | Thymol (0.24%) | Kollicoat MAE 30 | 80% | |
| | | DP (1.13%) | | |
| Ter (0.30%) | — | Kollicoat MAE 30 | 83% | |
| | | DP (1%) | | |
| Ter (0.60%) | — | Kollicoat MAE 30 | 82% | |
| | | DP (1%) | | |
| — | — | Kollicoat MAE 30 | 78% | |
| | | DP (1%) | | |

Abbreviation: Hex = 1,2-Hexanediol; Oct = 1,2-Octanediol; Ter = (−)-α-terpineol

TABLE 4
E. coli killing efficacy of the coating at different aging times
and bacteria exposure times and surface areas. The killing efficacy
data were from three repetitions. The spraying coverage was 50 m²/L (157
μL for 78.54 cm², 218 μL for 109.10 cm²). The well-plate lid model
was Cytiva Whatman ™ Clear Polystyrene Universal Lid
(Manufacturer: Cytiva 77041001). The Petri dish model was purchased
from VWR (polystyrene, catalog No. 25384-302).

| Surface | | | Bacteria | E. coli |
|---|---|---|---|---|
| area | | Aging time | exposure time | killing |
| (cm²) | Material | (day) | (min) | efficacy |
| 78.54 | Polystyrene | 0 | 10 | 99.43% |
| | (Petri dish) | | | |
| 78.54 | Polystyrene | 30 | 10 | 79% |
| | (Petri dish) | | | |
| 78.54 | Polystyrene | 30 | 90 | 91% |
| | (Petri dish) | | | |
| 109.10 | Polystyrene | 0 | 10 | 99.88% |
| | (well-plate lid) | | | |
| 109.10 | Polystyrene | 7 | 10 | 98.5% |
| | (well-plate lid) | | | |
| 109.10 | Polystyrene | 15 | 10 | 97.5% |
| | (well-plate lid) | | | |
| 109.10 | Polystyrene | 31 | 10 | 60% |
| | (well-plate lid) | | | |
| 33.50 | Ceramics (tile) | 0 | 10 | 95.8% |

TABLE 5
Candida albicans killing efficacy of the coating.

| | | Candida albicans |
|---|---|---|
| Form | Fungi exposure time | killing efficacy |
| Solid coating^{[1]} | 20 min | 60% |
| Solid coating^{[1]} | 4 h | 97.9% |
| Solid coating^{[1]} | 24 h | 79%^{[2]} |
| Liquid | 30 seconds | >=99.9999% |

^{[1]}The spraying coverage was 50 m²/L. The antifungal tests were done right after the coatings were dried and formed.^{[2]}

The number was lower than that of 4 h exposure time. It can make sense as the killing efficacy was relative to the control and a lot of the fungi also died on the control surfaces after 24 h given there was no nutrition.

### Example 11—Coating Removal

The transparent surface turned opaquer with more layers of coating applied (See FIG. 26). Removal procedure: After applying several layers of the coating formulation, it is recommended to remove them every 3-6 months before a new layer of coating is applied. The coating can be removed after an adequate amount of baking soda (sodium bicarbonate) aqueous solution is applied. To prepare the solution, 1.5 g of baking soda is dissolved in 100 mL of water. After preparation, the solution can be sprayed or wiped on the coated surface. The recommended spraying time is half the time used to spray coating formulation. For example, for 4 layers, spray the baking soda solution in the same way as the coating formulation (6 seconds per square meter) twice (or 12 seconds per square meter once). After applying the baking soda solution, wait for 30 seconds, wipe or rinse the surface with water to completely remove the coating. FIG. 26 shows a photo demonstrating the effect from the number of layers coated. The transparent surface turned opaquer with more layers of coating applied. The numbers at the top left corners of the photos are the number of layers coated. The spraying coverage was 50 m²/L for each layer.

### Example 12—Viscosity Measurements of the Coating Solution

The viscosity measurement was conducted on a TA Instruments DHR-2 Rheometer. The geometry was ROTOR CONICAL/DIN H/A-AL SMART-SWAP (Part number: 546011.901; Serial number: 114773), which was made of aluminum and had a bob diameter of 28.00 mm and a bob length of 42.01 mm. The cup had a diameter of 30.36 mm and was using a Peltier environmental system. The operating gap was 5917.1 μm. The volume of the liquid added was 25 mL. Flow sweep was selected as the method. Using the sample setup and at the same shear rate of 10 s⁻¹, the viscosity of water was measured to be 0.8789 mPa s, consistent with the literature value.

TABLE 6
Viscocity at a shear rate of 10 s⁻¹.

| Temperature (° C.) | Viscosity (mPa s) |
|---|---|
| 25 | 1.407 |
| 6.5 | 2.562 |

FIG. 27 show a graph demonstrating the viscosity the coating solution at 25° C. and 6.5° C. as a function of shear rate.

### Example 13—Scanning Electron Microscopy (SEM) Analysis

The coating solution was sprayed on small pieces (\tilde{ }5 mm) of glass, polystyrene plastics, both attached to SEM stubs by conductive tapes, and also sprayed on the stubs (made of aluminum) itself. The coverage was 50 m²/L. The samples were sputtered with 5 nm of Au. The SEM images were taken using the Hitachi S-4800 SEM. The detailed imaging conditions are summarized in the table 7:

TABLE 7
Parameters used for the SEM analysis

| Parameter | Value |
|---|---|
| Probe current | Normal |
| Focus mode | HR |
| Working distance | 8.0 mm |
| Focus depth | 1.5 |
| Accelerating voltage | 3.0 kV |

FIG. 28 shows a photo demonstrating the SEM images of the coated formulations on glass, plastic (polystyrene) and metal (aluminum) with 600×, 1200× and 2000× magnification.

### Example 14—The Release Kinetics of Thymol from the Coating

157 μL of the coating formulation was sprayed on each Petri dish. After the coatings were formed and dried, they were kept in a closed re-closable bag and aged for 0-30 days. At each time point, three Petri dishes (three repetitions) were taken out. 10 mL of ethanol was added to each Petri dish and put on a rocker to gently shake overnight to dissolve the coating. As some of the ethanol evaporated, the mass of the ethanol solution was weighed. The concentration of thymol in the solution was determined by the GC/MS peak area. The detailed conditions are summarized in the table 8:

TABLE 8
Parameters used for the GC-MS analysis. The
retention time of thymol was 9.0-9.1 min.

| Parameter | Value |
|---|---|
| GC | Agilent 7890B |
| MS | Agilent 5977B |
| Column | Agilent 19091S-433: 93.92873 |
| | HP-5MS 5% Phenyl Methyl Silox |
| | 0° C.-325° C. |
| | 30 m × 250 μm × 0.25 μm |
| Temperature | 0 min-2 min: hold at 50° C. |
| program | 2 min-16.667 min: 50° C. → 270° C., rate 15 |
| | ° C./min |
| | 16.667 min-18.667 min: hold at 270° C. |
| | 18.667 min-19.417 min: 270° C. → 315° C., |
| | rate 60° C./min |
| | 19.417 min-20.417 min: hold at 315° C. |
| | See FIG. 29. |
| Carrier gas | He |
| Flow rate | 1.2 mL/min |
| Pressure | 9.7853 psi |
| Average velocity | 39.923 cm/s |
| Holdup time | 1.2524 min |
| GC | Agilent 7890B |
| MS | Agilent 5977B |
| Column | Agilent 19091S-433: 93.92873 |
| | HP-5MS 5% Phenyl Methyl Silox |
| | 0° C.-325° C. |
| | 30 m × 250 μm × 0.25 μm |

TABLE 9
The release kinetic data of thymol from the coating.

| Aging time | % thymol left in the coating |
|---|---|
| (day) | relative to day 0 |
| 0 | 100.00 ± 22.34 |
| 7 | 21.45 ± 0.33 |
| 15 | 17.89 ± 4.01 |
| 21 | 19.80 ± 1.04 |
| 30 | 11.55 ± 2.12 |

### Example 15—The Evaluation of Spraying Ability at Different Pressure Levels

157 μL of the coating formulation was sprayed on each Petri dish. After the coatings were formed and dried, they were kept in a closed reclosable bag and aged for 0-30 days. At each time point, three Petri dishes.

The table in FIG. 30 demonstrate the time it takes for the Graco TC Pro SPRAYER to coat 1 m² of surface at 50 m^{2/}L at different pressure levels.

FIG. 31 shows a photo demonstrating the optical microscope photos of the coatings. Top two photos: Sprayed by Master Airbrush (Multi-purpose Gravity Feed Dual-action Airbrush Kit with 6 Foot Hose and a Powerful ⅕ hp Single Piston Quiet Air Compressor) with an amount of 50 m^{2/}L. Bottom two photos: Sprayed by Graco TC Pro Cordless Airless Paint Sprayer with a TC Pro 208 0.008 in. Tip at the pressure level 4 from a distance of about 1.5 m.

FIG. 32 shows a photo demonstrating the transmission electron microscopy (TEM) image of coating solution formulation showing that it contains nanoparticles.

FIG. 33 shows a graph demonstrating the particle size distribution by number of the coating solution made in the lab (lower line) and the one made by the Mexican manufacturer (large scale production, higher line) determined by dynamic light scattering (DLS). The former has a Z-average diameter of 148.9±0.9 nm and a PDI of 0.050±0.014. The latter has a Z-average diameter of 134.7±1.0 nm and a PDI of 0.016±0.011. The two were very close, indicating that the manufacturer was producing a product consistent with what we made in the lab.

### Example 16—Pilot Scale Manufacturing Procedure of the Coating Solution

Step 1. Dissolve thymol and 1,2--hexanediol in 96% ethanol by stirring for 2 mins at 1000 rmp in an industrial mixer,

Step 2. Add water and stir for 5 min at 1000 rpm in an industrial mixer.

Step 3. Add Kollicoat and stir to mix well in an industrial mixer at 300 rmp for 30 min.

Step 4. Let the mixture rest for 2 hours.

AH steps are performed at room temperature and in 10,000 L scale.

TABLE 10
Antimicrobial efficacy of the coating performed by third party certified lab in Mexico.

| | | | | Number | | |
|---|---|---|---|---|---|---|
| | | | | of CFU on | | |
| | | | | Veri | | |
| | | | | Shield | | |
| | | Microbe | Number | coated | | |
| | | exposure | of CFU on | Petri | Killing | |
| Microbe | State | time | control | dishes | efficacy | Method |
| Escherichia | Solid | 10 min | 8,900,000 | 7,000 | 99.921% | Our |
| coli | coating^{[1]} | | | | | standard |
| 11229 | | | | | | method |
| Staphylococcus | Solid | 10 min | 9,200,000 | 5,000,000 | 45.652% | Our |
| aureus | coating | | | | | standard |
| 6538 | | | | | | method |
| Saccharomyces | Solid | 4 h | 15,000 | 0 | 100% | Our |
| cerevisiae | coating | | | | | standard |
| 9763 | | | | | | method |
| Aspergillus | Solid | 4 h | 5,000 | 400 | 92% | Our |
| brasiliensis | coating | | | | | standard |
| 16404 | | | | | | method |
| Escherichia | Liquid | 30 s | | | 100% | NMX-BB- |
| coli | | | | | | 040-SCFI- |
| 11229 | | | | | | 1999 |
| Staphylococcus | Liquid | 30 s | | | 100% | NMX-BB- |
| aureus | | | | | | 040-SCFI- |
| 6538 | | | | | | 1999 |
| Saccharomyces | Liquid | 30 s | | | 100% | NMX-BB- |
| cerevisiae | | | | | | 040-SCFI- |
| 9763 | | | | | | 1999 |
| Aspergillus | Liquid | 30 s | | | 99.996% | NMX-BB- |
| brasiliensis | | | | | | 040-SCFI- |
| 16404 | | | | | | 1999 |
| Equine | Solid | 15 min | | | >99.9999% | E 1053- |
| Arteritis | coating | | | | | 97^{[2]} |
| virus ATCC | (10.9 | | | | | |
| VR-796 | m²/L, | | | | | |
| | 78.54 cm², | | | | | |
| | Petri dish) | | | | | |

^{[1]}All solid coatings in this table had a spraying coverage of 10.9 m²/L. The surface area was 78.54 cm² (10 cm diameter Petri dish). The Petri dish substrate was made of polystyrene.
^{[2]}Standard Test Method for Efficacy of Virucidal Agents Intended for Inanimate Environmental Surfaces

### Example 17—Finger Rubs on the Coating on Ceramics and Wood

The bottom parts of the substrates were coated, and the upper parts were coated. Contrast of the coated parts and the uncoated parts and a straight line at the border between the two could be seen under light. The researcher used his finger to rub the center part up and down across the border. Up and down were counted as two rubs. The numbers of rubs are shown at the top left corner of the photos. The black circle highlights the damage done to the coating at the end.

FIG. 34 shows the first photo shows the coated ceramic surface prior finger rub. The other photos show the impact of finger rubs on the coating, with the numbers of rubs are shown at the top left corner of the photos. The black circle highlights the damage done to the coating at the end.

FIG. 35 shows the first photo shows the coated wood surface prior finger rub. The other photos show the impact of finger rubs on the coating, with the numbers of rubs are shown at the top left corner of the photos. The red circle highlights the damage done to the coating at the end.

### Example 18—Coating of Apples for Preservation Purposes

1 mL of the coating solution was sprayed on an apple. The other apple was uncoated as a control. Both were stored under ambient conditions for 92 days.

FIG. 36 shows the first photo shows the uncoated and coated apple at day 1. The second photo after day 92.

FIG. 37 shows the first photo shows the uncoated apple sliced in the middle at day 92. The second photo the coated apple sliced in the middle at day 92.

### REFERENCES

1. Palaniappan, K.; Holley, R. A., International Journal of Food Microbiology 2010, 140 (2-3), 164-168.

2. Liu, Q.; Niu, H.; Zhang, W.; Mu, H.; Sun, C.; Duan, J., Letters in Applied Microbiology 2015, 60 (5), 421-30.

3. Zhou, F.; Ji, B.; Zhang, H.; Jiang, H.; Yang, Z.; Li, J.; Li, J.; Ren, Y.; Yan, W., Journal of Food Production 2007, 70 (7), 1704-1709. 4. Moon, H.; Rhee, M. S., International Journal of Food Microbiology 2016, 217, 35-41.

1. A water insoluble film forming antimicrobial, antibacterial, antiviral, antiparasitic and antifungal composition, comprising,
a) a film forming material selected from the group consisting of methacrylic acid-ethyl acrylate copolymer and methacrylic acid-acrylic ester copolymer,
b) one or more antimicrobial, antibacterial, antiviral, antiparasitic and antifungal compounds selected from the group consisting of thymol, benzethonium chloride, benzalkonium chloride, citric acid, lactic acid, carvacrol, 2-phenylphenol, chloroxylenol, 1,2-hexanediol, 1,2-octanediol, terpineol and 1,2-decanediol,
c) one or more solubilizing agents, which is a solvent, or a mixture of solvents selected from the group consisting of water and a low molecular weight alcohol, and
d) optionally, one or more plasticizer,
e) optionally, one or more surfactants or detergents.

2. The water insoluble film forming antimicrobial, antibacterial, antiviral, antiparasitic and antifungal composition according to claim 1, comprising
a) a film forming materials selected from the group consisting of ethyl cellulose, polyvinyl alcohol, or an acrylate polymers or co-polymers,
b) one or more antimicrobial, antibacterial, antiviral, antiparasitic and antifungal compounds selected from the group consisting of thymol, benzethonium chloride, benzalkonium chloride, citric acid, lactic acid and 1,2-hexanediol,
c) one or more solubilizing agents, which is a solvent, or a mixture of solvents selected from the group comprising consisting of water and a low molecular weight alcohol, such as ethanol and propanol,
d) optionally, one or more plasticizer, and
e) optionally, one or more surfactants or detergents.

3. The water insoluble film forming antimicrobial, antibacterial, antiviral, antiparasitic and antifungal composition according to claim 1, wherein the acrylate polymers or co-polymers is selected from the group consisting of methacrylic acid-ethyl acrylate copolymer and methacrylic acid-acrylic ester copolymer.

4. The water insoluble film forming antimicrobial, antibacterial, antiviral, antiparasitic and antifungal composition according to claim 1, wherein the the methacrylic acid-ethyl acrylate copolymer is Kollicoat MAE 30 DP™.

5. The water insoluble film forming antimicrobial, antibacterial, antiviral, antiparasitic and antifungal composition according to claims 1, wherein the pH of the liquid is below 6.

6. The water insoluble film forming antimicrobial, antibacterial, antiviral, antiparasitic and antifungal composition according to claim 1, whereby the film forming materials is methacrylic acid-ethyl acrylate copolymer at a ratio for methacrylic acid to ethyl acrylate of 5:1 to 1:5 .

7. The water insoluble film forming antimicrobial, antibacterial, antiviral, antiparasitic and antifungal composition according to claim 1, comprising
a) a film forming materials selected from methacrylic acid-ethyl acrylate copolymer at a ratio of 1:2 to 2:1,
b) one or more antimicrobial, antibacterial, antiviral, antiparasitic and antifungal compounds selected from the group consisting of thymol, 1,2-hexanediol and 1,2-octanediol,
c) one or more solubilizing agents, which is a solvent, or a mixture of solvents selected from the group consisting of water and ethanol,
d) optionally, one or more plasticizer, and e) optionally, one or more surfactants or detergents.

8. The water insoluble film forming antimicrobial, antibacterial, antiviral, antiparasitic and antifungal composition according to claim 1, comprising
a) a film forming materials selected from methacrylic acid-ethyl acrylate copolymer at a ratio of 1:1 to 1:1,
b) one or more antimicrobial, antibacterial, antiviral, antiparasitic and antifungal compounds selected from the group consisting of thymol and 1,2-hexanediol,
c) one or more solubilizing agents, selected from water and ethanol,
d) optionally, one or more plasticizer, and
e) optionally, one or more surfactants or detergents selected from polysorbate 80 and/or sodium laurate sulfate.

9. The water insoluble film forming antimicrobial, antibacterial, antiviral, antiparasitic and antifungal composition according to claim 1, whereby the concentrations of the antimicrobial, antibacterial, antiviral, antiparasitic and antifungal compound is 0.01-0.8 v/v % of 1,2-hexanediol and 0.01-0.8 w/v % of thymol, or 0.1-0.5 v/v % of 1,2-hexanediol and 0.1-0.5 w/v % of thymol, or 0.1-0.3 v/v % of 1,2-hexanediol and 0.1-0.3w/v % of thymol, or 0.25-3.2 v/v % 1,2-hexanediol and 0.15-0.25 w/v % thymol, whereby the percentages of the concetrations are relative to the total weight or volume of the composition.

10. The water insoluble film forming antimicrobial, antibacterial, antiviral, antiparasitic and antifungal composition according to claim 1, whereby the concentrations of the antimicrobial, antibacterial, antiviral, antiparasitic and antifungal compound is 0.30 w/v % 1,2-hexanediol and 0.24 w/v % thymol.

11. The water insoluble film forming antimicrobial, antibacterial, antiviral, antiparasitic and antifungal composition according to claim 1, wherein the surfactant or detergents or a mixture of surfactants or detergents is selected from the group consisting of glyceryl trioctanoate, dibutyl sebacate, triethyl citrate, triacetin, acetylated monoglycerides and diethyl phthalate or 1,2-hexanediol, glycerol, caprylic/capric triglycerides, glycerol monolaurate, anionic detergent and surfactant, carboxylates, carboxylate-based fluorosurfactants, or selected from the group consisting of nonionic and zwitter ionic surfactants and detergens, or cationinc detergents and surfactants.

12. The water insoluble film forming antimicrobial, antibacterial, antiviral, antiparasitic and antifungal composition according to claim 1 any one of the preceding claims, wherein the surfactant or detergents or a mixture of surfactants or detergents is selected from sodium lauryl sulfate and Polysorbate 80.

13. The water insoluble film forming antimicrobial, antibacterial, antiviral, antiparasitic and antifungal composition according to claim 1, further comprising one or more odour masking agent, selected from the group consisting of menthol, citronellal, citrus, lemon, and fragrances.

14. The water insoluble film forming antimicrobial, antibacterial, antiviral, antiparasitic and antifungal composition according to claim 1, further comprising one or more anti itching agent.

15. The water insoluble film forming antimicrobial, antibacterial, antiviral, antiparasitic and antifungal composition according to claim 1, further comprising one or more anti-inflammatory agent.

16. (canceled)

17. (canceled)

18. (canceled)

19. (canceled)

20. (canceled)

21. (canceled)

22. (canceled)

23. (canceled)
